SEQUENCE LISTING (125 sequences)

SEQ ID NO: 1 - DNA, 19 bp, Artificial
  SgRNA
tgtactctctgaggtgctc

SEQ ID NO: 2 - DNA, 19 bp, Artificial
  SgRNA
acgcagataagaaccagtt

SEQ ID NO: 3 - DNA, 19 bp, Artificial
  SgRNA
catcaagtcagccatcagc

SEQ ID NO: 4 - DNA, 19 bp, Artificial
  SgRNA
gagtcaccctcctggaaac

SEQ ID NO: 5 - DNA, 20 bp, Artificial
  SgRNA
ccgacagcgcccgaccccaa

SEQ ID NO: 6 - DNA, 20 bp, Artificial
  SgRNA
ccccgggcgagcgggatggg

SEQ ID NO: 7 - DNA, 20 bp, Artificial
  SgRNA
gccagccaatgagcgccgcc

SEQ ID NO: 8 - DNA, 20 bp, Artificial
  SgRNA
cccgcgtgcacccccagggg

SEQ ID NO: 9 - DNA, 20 bp, Artificial
  SgRNA
ggataggtaaaggggcgcgt

SEQ ID NO: 10 - DNA, 20 bp, Artificial
  SgRNA
gaaggtcactgcgccccgac

SEQ ID NO: 11 - DNA, 20 bp, Artificial
  SgRNA
gcgcagagcgagagcgctcg

SEQ ID NO: 12 - DNA, 20 bp, Artificial
  SgRNA
gtctgactatggggcggagt

SEQ ID NO: 13 - DNA, 19 bp, Artificial
  SgRNA
cctgggctccggggcgttt

SEQ ID NO: 14 - DNA, 19 bp, Artificial
  SgRNA
ggcccctgcggccaccccg

SEQ ID NO: 15 - DNA, 19 bp, Artificial
  SgRNA
ctccctccctgcccggtag

SEQ ID NO: 16 - DNA, 19 bp, Artificial
  SgRNA
aggtttggaaagggcgtgc

SEQ ID NO: 17 - DNA, 34 bp, Artificial
  Primer
ccgacaccgcggagtccccctgtatgggttcacc

SEQ ID NO: 18 - DNA, 81 bp, Artificial
  Primer
ccgacaggcgcgcccaactttgcgtttctttttcgggctagccttggatccggcaattggcttcaggtccacgacgacgac

SEQ ID NO: 19 - DNA, 54 bp, Artificial
  Primer
gaattcctcgagctcaagcttgcgggactgcagatggacaagaagtattctatc

SEQ ID NO: 20 - DNA, 44 bp, Artificial
  Primer
aattcgtcttgagttggcgcgcccccggtagaatttacctttct

SEQ ID NO: 21 - DNA, 20 bp, Artificial
  Primer
ccacatgccaagcactttac

SEQ ID NO: 22 - DNA, 21 bp, Artificial
  Primer
gatcctgggccacatctataa

SEQ ID NO: 23 - DNA, 20 bp, Artificial
  Primer
ccctgtcaggagggacagat

SEQ ID NO: 24 - DNA, 20 bp, Artificial
  Primer
ggctcaccggaagcatgaat

SEQ ID NO: 25 - DNA, 20 bp, Artificial
  Primer
tgttccctccacctggaata

SEQ ID NO: 26 - DNA, 20 bp, Artificial
  Primer
gggaaaatccaaagcaggat

SEQ ID NO: 27 - DNA, 20 bp, Artificial
  Primer
gttagagcgttggggacctt

SEQ ID NO: 28 - DNA, 21 bp, Artificial
  Primer
cacatgcagagaactgagctg

SEQ ID NO: 29 - DNA, 20 bp, Artificial
  Primer
ttctctgcatgtgacctccc

SEQ ID NO: 30 - DNA, 20 bp, Artificial
  Primer
acacactcacagagggttgg

SEQ ID NO: 31 - DNA, 20 bp, Artificial
  Primer
gctgggctcctccttgtact

SEQ ID NO: 32 - DNA, 20 bp, Artificial
  Primer
gctgctgcccataaagtagc

SEQ ID NO: 33 - DNA, 22 bp, Artificial
  Primer
caggtgaacagagaggtgtaac

SEQ ID NO: 34 - DNA, 25 bp, Artificial
  Primer
ggctatttcaccaatttccctattc

SEQ ID NO: 35 - DNA, 23 bp, Artificial
  Primer
catggtgaaaccctgtctctatt

SEQ ID NO: 36 - DNA, 22 bp, Artificial
  Primer
caattctcctgccttagctacc

SEQ ID NO: 37 - DNA, 22 bp, Artificial
  Primer
tgtagtaagcacagtggaagtg

SEQ ID NO: 38 - DNA, 22 bp, Artificial
  Primer
gagagggttcagtgggataaac

SEQ ID NO: 39 - DNA, 21 bp, Artificial
  Primer
gagaccagcctgaccaatatg

SEQ ID NO: 40 - DNA, 18 bp, Artificial
  Primer
ccgggttcaagcgattct

SEQ ID NO: 41 - DNA, 21 bp, Artificial
  Primer
ctgagggaacacagctacatc

SEQ ID NO: 42 - DNA, 23 bp, Artificial
  Primer
ggagacacttcactgagaatagg

SEQ ID NO: 43 - DNA, 20 bp, Artificial
  Primer
ccactacgcctggctaattt

SEQ ID NO: 44 - DNA, 19 bp, Artificial
  Primer
agatgcgaggtcaggagat

SEQ ID NO: 45 - DNA, 20 bp, Artificial
  Primer
ccaccacacccagctaattt

SEQ ID NO: 46 - DNA, 20 bp, Artificial
  Primer
gcctgtaatcccagcacttt

SEQ ID NO: 47 - DNA, 21 bp, Artificial
  Primer
cgggtagaggagtctgaatct

SEQ ID NO: 48 - DNA, 21 bp, Artificial
  Primer
ctagtgtgcttgagtgacagg

SEQ ID NO: 49 - DNA, 17 bp, Artificial
  Primer
atcccgctggagtcgat

SEQ ID NO: 50 - DNA, 17 bp, Artificial
  Primer
gcgccaccctttccttt

SEQ ID NO: 51 - DNA, 20 bp, Artificial
  Primer
acagactcagagagaaccca

SEQ ID NO: 52 - DNA, 17 bp, Artificial
  Primer
gcctccgcaccatactc

SEQ ID NO: 53 - DNA, 17 bp, Artificial
  Primer
tggacgacatgcccaac

SEQ ID NO: 54 - DNA, 17 bp, Artificial
  Primer
gccgctcaccttgaagt

SEQ ID NO: 55 - DNA, 20 bp, Artificial
  Primer
ccgtgctgacctccaaatac

SEQ ID NO: 56 - DNA, 18 bp, Artificial
  Primer
gcccactcagactttatt

SEQ ID NO: 57 - DNA, 21 bp, Artificial
  Primer
catgcctgtaaacccacctac

SEQ ID NO: 58 - DNA, 21 bp, Artificial
  Primer
ggagtgcaatggtgtgatctt

SEQ ID NO: 59 - DNA, 17 bp, Artificial
  Primer
cagggagcaggctgaag

SEQ ID NO: 60 - DNA, 20 bp, Artificial
  Primer
gggatggtactgaggagaaa

SEQ ID NO: 61 - DNA, 19 bp, Artificial
  Primer
ccaggaagccctcagacta

SEQ ID NO: 62 - DNA, 22 bp, Artificial
  Primer
ccatcacacaagtacacacaga

SEQ ID NO: 63 - DNA, 21 bp, Artificial
  Primer
cgggtagaggagtctgaatct

SEQ ID NO: 64 - DNA, 21 bp, Artificial
  Primer
ctagtgtgcttgagtgacagg

SEQ ID NO: 65 - DNA, 17 bp, Artificial
  Primer
atcccgctggagtcgat

SEQ ID NO: 66 - DNA, 17 bp, Artificial
  Primer
gcgccaccctttccttt

SEQ ID NO: 67 - DNA, 20 bp, Artificial
  Primer
acagactcagagagaaccca

SEQ ID NO: 68 - DNA, 17 bp, Artificial
  Primer
gcctccgcaccatactc

SEQ ID NO: 69 - DNA, 17 bp, Artificial
  Primer
tggacgacatgcccaac

SEQ ID NO: 70 - DNA, 17 bp, Artificial
  Primer
gccgctcaccttgaagt

SEQ ID NO: 71 - DNA, 20 bp, Artificial
  Primer
ccgtgctgacctccaaatac

SEQ ID NO: 72 - DNA, 18 bp, Artificial
  Primer
gcccactcagactttatt

SEQ ID NO: 73 - DNA, 19 bp, Artificial
  Primer
atatgggtggtgggtaggt

SEQ ID NO: 74 - DNA, 24 bp, Artificial
  Primer
tttcatctagactgtgcctgtatc

SEQ ID NO: 75 - DNA, 18 bp, Artificial
  Primer
tctgcctgcgtttgtgat

SEQ ID NO: 76 - DNA, 22 bp, Artificial
  Primer
cagacctccattcttcctgatg

SEQ ID NO: 77 - DNA, 20 bp, Artificial
  Primer
gcctgtaatcccagcacttt

SEQ ID NO: 78 - DNA, 19 bp, Artificial
  Primer
caccacacccggctaattt

SEQ ID NO: 79 - DNA, 20 bp, Artificial
  Primer
tacagataggctgggagtgg

SEQ ID NO: 80 - DNA, 21 bp, Artificial
  Primer
caggacgtggaggatctctaa

SEQ ID NO: 81 - DNA, 22 bp, Artificial
  Primer
gtgtgtgtgtgtgtgtgtattg

SEQ ID NO: 82 - DNA, 21 bp, Artificial
  Primer
cccatagagatgctgttagcc

SEQ ID NO: 83 - DNA, 20 bp, Artificial
  Primer
gggtgtgcccatgtgtatta

SEQ ID NO: 84 - DNA, 22 bp, Artificial
  Primer
cggatgaggacatacacttagc

SEQ ID NO: 85 - DNA, 18 bp, Artificial
  Primer
agccagagagggtgagag

SEQ ID NO: 86 - DNA, 22 bp, Artificial
  Primer
cccatcactaacaggagacaag

SEQ ID NO: 87 - DNA, 20 bp, Artificial
  Primer
agtgtcgggaatgtgtgtag

SEQ ID NO: 88 - DNA, 22 bp, Artificial
  Primer
gggtagtggataagctgtagtg

SEQ ID NO: 89 - DNA, 20 bp, Artificial
  Primer
cccattctcctctgacctct

SEQ ID NO: 90 - DNA, 20 bp, Artificial
  Primer
catgtgtcctctgccacttt

SEQ ID NO: 91 - DNA, 17 bp, Artificial
  Primer
taaggccagctcctcca

SEQ ID NO: 92 - DNA, 22 bp, Artificial
  Primer
actaccacctagagagcatagg

SEQ ID NO: 93 - DNA, 17 bp, Artificial
  Primer
gagtgggccacgaagca

SEQ ID NO: 94 - DNA, 17 bp, Artificial
  Primer
tccgcctcctctggaca

SEQ ID NO: 95 - DNA, 17 bp, Artificial
  Primer
gggaagcgggaagacag

SEQ ID NO: 96 - DNA, 22 bp, Artificial
  Primer
cagacagaaagaaggaccagag

SEQ ID NO: 97 - DNA, 20 bp, Artificial
  Primer
gggtccttgagttagttgct

SEQ ID NO: 98 - DNA, 21 bp, Artificial
  Primer
caatcacacaggcttggaaag

SEQ ID NO: 99 - DNA, 20 bp, Artificial
  Primer
tccaccttgctgtctcattc

SEQ ID NO: 100 - DNA, 22 bp, Artificial
  Primer
agggttctaacctttggttctt

SEQ ID NO: 101 - DNA, 20 bp, Artificial
  Primer
ccactgctgagtttggtagg

SEQ ID NO: 102 - DNA, 22 bp, Artificial
  Primer
gggacaggatggtgtcattaag

SEQ ID NO: 103 - DNA, 19 bp, Artificial
  Primer
gcacacatcctcgacagtt

SEQ ID NO: 104 - DNA, 20 bp, Artificial
  Primer
atcaccatgggtcgcataag

SEQ ID NO: 105 - DNA, 21 bp, Artificial
  Primer
cctactatgcacctggctttc

SEQ ID NO: 106 - DNA, 21 bp, Artificial
  Primer
ggcatctcgatgtgtcttctt

SEQ ID NO: 107 - DNA, 20 bp, Artificial
  Primer
tgcctccaggtgtagtaaga

SEQ ID NO: 108 - DNA, 21 bp, Artificial
  Primer
ccagcagcttcttgcatttag

SEQ ID NO: 109 - DNA, 21 bp, Artificial
  Primer
tcctaattggtctccctcctt

SEQ ID NO: 110 - DNA, 22 bp, Artificial
  Primer
gcttgagcggaagagaagaata

SEQ ID NO: 111 - DNA, 21 bp, Artificial
  Primer
agcagcagagaagtgaagaag

SEQ ID NO: 112 - DNA, 21 bp, Artificial
  Primer
tctttgtgcctttcccactaa

SEQ ID NO: 113 - DNA, 20 bp, Artificial
  Primer
tcggagacttcaggtgagat

SEQ ID NO: 114 - DNA, 20 bp, Artificial
  Primer
ggtgctgagtcaaggaaagt

SEQ ID NO: 115 - DNA, 23 bp, Artificial
  Primer
gggacagaggagtattgaaagtc

SEQ ID NO: 116 - DNA, 20 bp, Artificial
  Primer
ccctttccaaacctctccaa

SEQ ID NO: 117 - DNA, 18 bp, Artificial
  Primer
tcctattggcctcggact

SEQ ID NO: 118 - DNA, 17 bp, Artificial
  Primer
gcgccctgggctattta

SEQ ID NO: 119 - DNA, 20 bp, Artificial
  Primer
ccgcctgagcaaagtaaatg

SEQ ID NO: 120 - DNA, 19 bp, Artificial
  Primer
cgatatagcggatggcgtt

SEQ ID NO: 121 - DNA, 20 bp, Artificial
  Primer
atctcctacccacccgtaat

SEQ ID NO: 122 - DNA, 18 bp, Artificial
  Primer
aggcccgaacaccaattc

SEQ ID NO: 123 - protein, 14 aa, Oryza sativa
YFPEEVVEHIFSFL

SEQ ID NO: 124 - protein, 14 aa, Arabidopsis thaliana
SFPEEVLEHVFSFI

SEQ ID NO: 125 - protein, 14 aa, Artificial
  Variant
YFPEKVVKHIFSFL

(19) United States
(12) Patent Application Publication
(10) Pub. No.: US 2019/0256827 A1
(43) Pub. Date: Aug. 22, 2019

(54) EPIGENOME EDITING SYSTEMS AND METHODS

(71) Applicant: STC.UNM, Albuquerque, NM (US)

(72) Inventors: Fu-Sen Liang, Albuquerque, NM (US); Tingjun Chen, Albuquerque, NM (US); Dan Gao, Albuquerque, NM (US)

(21) Appl. No.: 16/227,279

(22) Filed: Dec. 20, 2018

Related U.S. Application Data
(60) Provisional application No. 62/611,613, filed on Dec. 29, 2017.

(51) Int. Cl. C12N 9/10; C12N 9/22; C12N 15/11; C12N 15/85

(52) U.S. Cl. CPC C12N 9/1029; C12N 9/22; C12N 2310/20; C12Y 203/01048; C12N 15/85; C12N 15/11

(57) ABSTRACT

An epigenome editing system generally includes three exogenous coding regions. The first exogenous coding region encodes a first polypeptide that includes an epigenetic editing domain and a domain that, in the presence of an inducer, forms a complex with a second polypeptide. The second exogenous coding region encodes the second polypeptide, which includes a dCas9 domain and a domain that, in the presence of the inducer, forms a complex with the first polypeptide. The third exogenous coding region encodes an sgRNA sequence that targets the dCas9 domain to a genomic locus.

### CROSS-REFERENCE TO RELATED APPLICATION

This application claims priority to U.S. Provisional Patent Application No. 62/611,613, filed Dec. 29, 2017, which is incorporated herein by reference in its entirety.

### GOVERNMENT FUNDING

This invention was made with government support under HG008776 awarded by National Institutes of Health. The government has certain rights in the invention.

### SUMMARY

This disclosure describes, in one aspect, a cell that includes a polynucleotide that encodes an epigenome editing system. Generally, the polynucleotide includes three exogenous coding regions. The first exogenous coding region encodes a first polypeptide that includes an epigenetic editing domain and a domain that, in the presence of an inducer, forms a complex with a second polypeptide. The second exogenous coding region encodes the second polypeptide, which includes a dCas9 domain and a domain that, in the presence of the inducer, forms a complex with the first polypeptide. The third exogenous coding region encodes an sgRNA sequence that targets the dCas9 domain to a genomic locus.

In some embodiments, the cell further includes the inducer.

In some embodiments, the inducer can include abscisic acid (ABA), gibberellic acid (GA), rapamycin, or other heterodimerizing inducers.

In some embodiments, the epigenetic editing domain can include a histone-modifying domain, a DNA methylating domain, a DNA demethylating domain, or a chromatin remodeling domain.

In another aspect, this disclosure describes a method of performing epigenetic editing of a genomic locus. Generally, the method includes introducing into cells any embodiment of the epigenome editing system summarized above, providing the inducer in an amount effective to induce the first polypeptide to form a complex with the second polypeptide, allowing the sgRNA to target the genomic locus, thereby delivering the epigenetic editing domain to the genomic locus, and allowing the epigenetic editing domain to modify the genomic locus.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. An exemplary chemical-induced or light-inducible integrated CRISPR/CIP system to study the function of histone PTMs.

FIG. 2. ABA-induced luciferase expression using integrated CRISPR/CIP technology. Transcription activator (VP80) was recruited to dCas9 that binds sgRNA targeted to the promoter region of the luciferase coding gene.

FIG. 3. Testing ABA-inducible dCas9-mediated recruitment of P300 and installation of H3K27Ac. (A) Constructs of dCas9-PYL-HA and FLAG-ABI-P300. (B) The sites targeted by sgRNAs (stars) and locations probed by qPCR amplicon (circles) at the IL1RN locus. (C) Enrichment of dCas9-PYL-HA at the IL1RN locus with or without sgRNAs. (D) Enrichment of FLAG-ABI-P300 at the IL1RN locus with or without ABA treatment. (E) Enrichment of FLAG-ABI-P300 H3K27Ac at the IL1RN locus with or without ABA treatment. (F) Levels of IL1RN mRNA. ** represents P<0.01 compared with control group. Fold changes were calculated by comparing to cells treated without sgRNA (C), with DMSO (D,E) or transfected with dCas9-PYL (F). Error bars represent ±s.e.m. from biological replicates (n=5).

FIG. 4. p300-related plasmid constructs.

FIG. 5. SUV39H1-related lentivirus and adenovirus plasmid constructs.

FIG. 6. Two histone-modification-related lentivirus and adenovirus plasmid constructs.

FIG. 7. P300 mutant that lacks the acetyltransferase activity did not increase IL1RN mRNA expression upon ABA addition. dCAs9-PYL-HA, FLAG-ABI-P300, FLAG-ABI-P300^{D1399Y} and IL1RN sgRNAs were transfected into HEK293T cells for 24 h. Cells were then treated with 100 μM of ABA for 24 hours before been harvested to analyze IL1RN mRNA expression using q-PCR. *** represents P<0.001 compared with DMSO group. The fold changes were calculated by comparing the sample under each treatment condition to the sample from cells transfected with dCAs9-PYL-HA. Error bars represent ±s.e.m. from biological replicates (n=6).

FIG. 8. Small molecule induced acetylation of H3K27 at the GRM2 locus. (A) The sites targeted by sgRNAs (stars) and locations probed by qPCR amplicon (circles) at the human GRM2 locus. (B) Enrichment of dCas9-PYL-HA at the GRM2 locus with or without sgRNAs. (C) Enrichment of FLAG-ABI-P300 at the GRM2 locus with or without ABA treatment for 48 hours. (D) Enrichment of H3K27Ac at the GRM2 locus with or without ABA treatment for 48 hours. (E) Levels of GRM2 mRNA under different conditions. *** represents P<0.001 compared with DMSO group. The fold changes were calculated by comparing the sample under each treatment condition to the sample from cells treated without sgRNA (B) or with DMSO (C-E). Error bars represent ±s.e.m. from biological replicates (n=5).

FIG. 9. Small molecule induced acetylation of H3K27 at the HBA locus. (A) The sites targeted by sgRNAs (stars) and locations probed by qPCR amplicon (circles) at the human HBA locus. Human HBA gene locus contains two copies of repeating promoter (1 kb upstream of TSS) and gene body. (B) Enrichment of dCas9-PYL-HA at the HBA locus with or without sgRNAs. (C) Enrichment of FLAG-ABI-P300 at the HBA locus with or without ABA treatment for 48 hours. (D) Enrichment of H3K27Ac at the HBA locus with or without ABA treatment for 48 hours. (E) Levels of HBA mRNA under different conditions. *** represents P<0.001 compared DMSO group. The fold changes were calculated by comparing the sample under each treatment condition to the sample from cells treated without sgRNA (B) or with DMSO (C-E). Error bars represent ±s.e.m. from biological replicates (n=5).

FIG. 10. Small molecule induced acetylation of H3K27 at the MYOD1 locus. (A) The sites targeted by sgRNAs (stars) and locations probed by qPCR amplicon (circles) at the human MYOD1 locus. (B) Enrichment of dCas9-PYL-HA at the MYOD1 locus with or without sgRNAs. (C) Enrichment of FLAG-ABI-P300 at the MYOD1 locus with or without ABA treatment for 48 hours. (D) Enrichment of H3K27Ac at the MYOD1 locus with or without ABA treatment for 48 hours. (E) Levels of MYOD1 mRNA under different conditions. *** represents P<0.001 compared with DMSO group. The fold changes were calculated by comparing the sample under each treatment condition to the sample from cells treated without sgRNA (B) or with DMSO (C-E). Error bars represent ±s.e.m. from biological replicates (n=5).

FIG. 11. Expression of six off-target genes when induced by ABA using sgRNAs designed for IL1RN. dCas9-PYL-HA, FLAG-ABI-P300 and IL1RN sgRNAs were transfected into HEK293T cells for 24 hours. Cells were then treated with 100 μM of ABA for another 24 hours followed by the analysis of mRNA levels of six off-target genes using q-PCR. The fold changes were calculated by comparing ABA treated cells to cells treated with DMSO. Error bars represent ±s.e.m. from biological replicates (n=6).

FIG. 12. ABA-induced IL1RN expression levels when using sgRNAs designed for GRM2, HBA or MYOD1 HEK293T cells were transfected with dCas9-PYL-HA, FLAG-ABI-P300 and indicated sgRNAs for 24 hours, followed by 24-hour ABA treatment. The levels of mRNA were determined by qPCR. (A) sgRNAs for GRM2 were used and then the levels of both GRM2 and IL1RN mRNAs were determined. (B) sgRNAs for HBA were used and then the levels of both HBA and IL1RN mRNAs were determined. (C) sgRNAs for MYOD1 were used and then the levels of both MYOD1 and IL1RN mRNAs were determined. *** represents P<0.001 compared with DMSO group. The fold changes were calculated by comparing ABA treated cells to cells treated with DMSO. Error bars represent ±s.e.m. from biological replicates (n=5).

FIG. 13. The time course of the enrichment of dCas9-PYL-HA at the IL1RN locus at indicated time points after transfection. Fold changes were calculated by comparing to cells not treated with sgRNA. Error bars represent ±s.e.m. from biological replicates (n=6).

FIG. 14. The time course of H3K27 acetylation and IL1RN expression. (A) Enrichment of FLAG-ABI-P300 at the IL1RN locus at indicated time points after ABA treatment. (B) Enrichment of H3K27Ac at the IL1RN locus at indicated time points after ABA treatment. (C) Levels of IL1RN mRNA at indicated time points after ABA treatment. ** represents P<0.01 compared with DMSO group; *** represents P<0.001 compared with DMSO group. Fold changes were calculated by comparing to cells treated with DMSO (A, B, C). (D) The time required to reach 50% maximum enrichments of FLAG-ABI-P300 (at primer 5 locus) and H3K27Ac (at primer 1 locus) at the IL1RN locus, and IL1RN mRNA expression. Error bars represent ±s.e.m. from biological replicates (n=6 in A, B, and D; n=8 in C).

FIG. 15. Distance dependency to reach 50% maximum enrichment of H3K27Ac at different probed locations at the IL1RN locus. (A) The time to reach 50% maximum enrichment of H3K27Ac at up-stream probed loci. (B) The time to reach 50% maximum enrichment of H3K27Ac at down-stream loci. Primer 1 and 9 probed loci are farthest from the FLAG-ABI-P300 localization site and primer 3 and 7 are closer.

FIG. 16. The reversibility of H3K27 acetylation and IL1RN expression. (A) Enrichment of FLAG-ABI-P300 at primer 5 locus at the IL1RN locus at indicated time points after ABA treatment with or without washing. (B) Enrichment of H3K27Ac at primer 1 locus at the IL1RN locus at indicated time points after ABA treatment with or without washing. (C) Reversing rate of H3K27Ac at the IL1RN locus after ABA removal. (D) Levels of IL1RN mRNA at indicated time points after ABA treatment, or after washing. ### represents P<0.001 compared with 48 hours; *** represents P<0.001 compared with 24 hours; \hat{ }\hat{ }\hat{ } represents P<0.001 compared with 12 hours. Fold enrichments were calculated by comparing to DMSO controls. Error bars represent ±s.e.m. from biological replicates (n=6 in A and B; n=8 in c).

FIG. 17. The reversing dynamics of localized FLAG-ABI-P300 and installed H3K27Ac. HEK293T cells were transfected with dCas9-PYL-HA, FLAG-ABI-P300, and IL1RN sgRNAs for 24 hours followed by 12 hours, 24 hours, or 48 hours of ABA treatment. ABA was removed by washing and cells were then harvested at indicated time points after washing for ChIP-qPCR analysis. The enrichments of FLAG-ABI-P300 (A, C, E) and H3K27Ac (B, D, F) at the IL1RN locus at indicated time points after washing were determined by comparing to cell treated with DMSO. (n=4).

FIG. 18. Time-dependent induction and reversibility of H3K27 acetylation at the GRM2 locus. HEK293T cells were transfected with dCas9-PYL-HA, FLAG-ABI-P300 and GRM2 sgRNAs for 24 hours and then treated with ABA for four hours, eight hours, 16 hours, or 24 hours, followed by washing to remove ABA and continued culture without ABA for another 24 hours. Transfected and treated cells were harvested at indicated time points for analysis of H3K27Ac enrichment at the GRM2 locus (at Primer 7 locus). * represents P<0.05 compared with DMSO group; ** represents P<0.01 compared with DMSO group; *** represents P<0.001 compared with DMSO group. The fold changes were calculated by comparing to cells treated with DMSO. Error bars represent ±s.e.m. from biological replicates (n=2).

FIG. 19. Precise temporal control of H3K27 acetylation at the IL1RN locus. HEK293T cells were transfected with dCas9-PYL-HA, FLAG-ABI-P300 and IL1RN sgRNAs for 24 hours and then treated with ABA for 12 hours, followed by washing to remove ABA. After an additional 12-hour incubation without ABA, cells were treated with another dose of ABA for 12 hours. (A) Transfected and treated cells were harvested at indicated time points for analysis of H3K27Ac enrichment at the IL1RN locus (at Primer 1 locus). (B) Transfected and treated cells were harvested at indicated time points for analysis of the level of IL1RN mRNA. The fold changes were calculated by comparing to cells treated with DMSO. Error bars represent ±s.e.m. from biological replicates (n=4 in A; n=6 in B).

FIG. 20. Auxin-based engineered CIP system. Auxin induces dimerization of auxin-inducible degron (AID) and transport inhibitor response 1 (TIR1) (A) Normal auxin-dependent TIR1-mediated AID degradation process. (B) Engineered auxin-dependent TIR1*-AID dimerization without degradation.

FIG. 21. Analysis of auxin-based CIP system. (A) Sequence alignment between Oryza sativa TIR sequence (SEQ ID NO:123) and Arabidopsis thaliana TIR sequence (SEQ ID NO:124). (B) EGFP is expressed only when a functional transcriptional activator was reconstituted by IAA from two split halves (a DNA binding domain, DBD, and an activation domain, AD, each linked to an IAA-dimerizable protein, TIR* and AID). This indicated that a working IAA-inducible CIP system was successfully created without degrading the AID fusion protein. (C) An auxin-based CIP system was co-transfected into HEK293 cells with an ABA-based system. Only the proper combination of inducer with corresponding binding proteins could induce luciferase expression, suggesting that the IAA and ABA systems can co-exist in a single cell and be independently induced. (D) The auxin-based CIP system is reversible. Luciferase expression decreased after the IAA inducer is removed by washing.

FIG. 22. Schematic illustration of an embodiment in which the presence of two inducers (ABA and auxin (IAA), as illustrated) are both required to initiate epigenetic change (histone modification, as illustrated).

FIG. 23. Schematic illustration of a multiple-inducer embodiment of the system in the context of epigenetic editing through DNA methylation and/or chromatin remodeling. As illustrated, a two-inducer system can be designed so that each inducer can induce a different epigenetic activity. The system can be activated using either inducer individually, using both inducers simultaneously, or using the two inducers sequentially.

FIG. 24. H3K27Ac level can be controlled using a light-inducible CIP technology. ABA was chemically modified to include a photo-cleavable group (e.g. 4,5-dimethoxy-2-nitrobenzyl, DMNB or [7-(diethylamino)coumarin-4-yl]methyl, DEACM) that masked ABA dimerization function and could be removed upon irradiation with a specific wavelength of light. (A) Enrichment of H3K27Ac at the IL1RN locus with UV light, with or without ABA treatment. (B) Levels of IL1RN mRNA under different conditions. mRNA expression with 20 μM ABA-DMNB (caged ABA) was at levels of negative control (DMSO). mRNA levels increased when ABA was uncaged by exposure to UV light. * represents P<0.001 compared with DMSO group.

### DETAILED DESCRIPTION OF ILLUSTRATIVE EMBODIMENTS

Epigenetics studies the heritable changes in gene activity that occur without genetic alterations but mainly through modifications of DNA or of histone proteins. Chromatin consists of nucleosomes, each contains a hi stone octamer core with DNA wrapping around it. Four histones (H2A, H2B, H3, and H4) form the octamer and each histone has an amino-terminal tail region of 25-40 amino acid residues that can be modified (e.g., acetylation, methylation, phosphorylation, etc.) to alter the compactness of chromatin and/or change the chromatin surface to modulate its association with chromatin-binding proteins. In addition to histone modification, other epigenetic pathways such as, for example, DNA methylation and ATP-dependent chromatin remodeling also regulate the status of chromatin. These changes in chromatin structure are considered to regulate the activity of associated genes. Epigenetic dysregulation has been linked to erroneous gene expression patterns that lead to the pathogenesis of various human diseases including cancers. Different combinations of histone posttranslational modifications (PTMs) are also proposed to form the “histone code” that guides gene expression. Different chromatin-modifying and chromatin-remodeling proteins have been discovered to read, write, and/or erase these histone PTMs. Certain histone PTMs correlations with gene activity. For example, the H3K4me3 PTM (the trimethylated lysine 4 on the H3 tail) is commonly associated with active genes and the H3K9me3 is associated with repressed genes and heterochromatin. Also, there may be synergistic or antagonistic effects between different histone PTMs. However, it is difficult to establish whether the presence of a certain histone PTM at specific gene locus is merely the by-product of gene activity or indeed has causal effects on gene expression. Also, it is still challenging to investigate the interactions or crosstalk between histone PTMs or between different epigenetic pathways. To effectively address these issues, methods that allow the editing or creation of a specific local epigenetic environment in living cells are needed.

This disclosure describes such methods. The methods involve integrating genome editing CRISPR technology with methods that involve chemically-induced proximity (CIP). The CIP technology has been developed to regulate biological processes using small molecule inducers that each dimerizes two unique inducer-binding protein fragments, which can in turn be fused individually to any two proteins of interest (POIs). The proteins of interest can be selected to temporally control desired downstream biological events using the chemical inducer.

While described herein in the context of an exemplary embodiment in which CIP system uses the plant abscisic acid signaling pathway as the dimerizing inducer, the systems and methods described herein can provide use any suitable dimerization inducers. In the plant abscisic acid (ABA) stress signaling pathway, the plant hormone ABA chemically induces dimerization of PYL and ABI proteins. Other exemplary CIP systems include using rapamycin (Rap) to dimerize FKBP and FRB, gibberellic acid (GA) to dimerize GID1 and GAI, fusicossin (FC) to dimerize 14-3-3 and CT52, auxin (indole-3-acetic acid, IAA) to dimerize auxin response factors (ARFs) such as, for example, TIR1 and AID, or other natural and synthetic ligands that can dimerize two fusion proteins of interest. In some embodiments, as described in more detail below, the CIP system described herein can include a modified version of at least one of the components of the CIP to system.

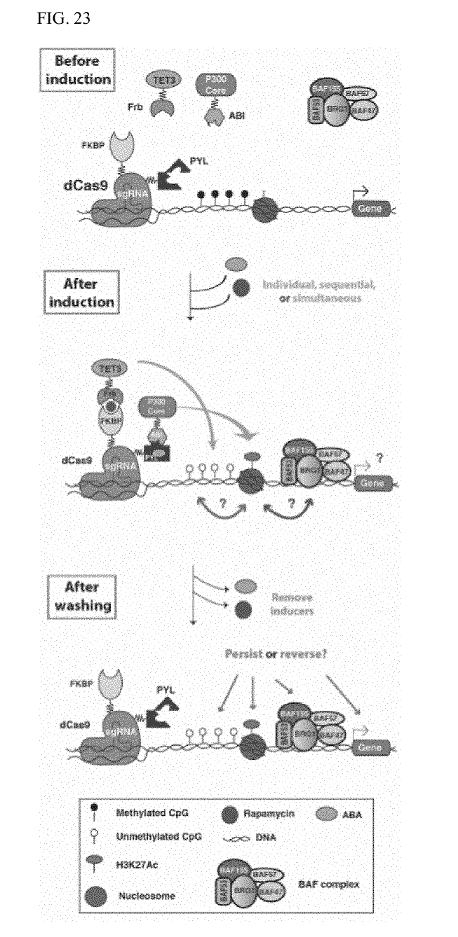

Also, while sometimes described herein in the context of exemplary embodiments in which the epigenetic editing involves histone modification, the systems and methods described herein can involve other epigenetic modifications. Exemplary alternative epigenetic modifications include, for example, DNA methylation, DNA, demethylation, or chromatin remodeling. Thus, the epigenetic editing domain can include a histone-modifying enzyme, a methyltransferase (e.g., DNMT), a demethylase (TET), a chromatin remodeling complexes (e.g., BAF and/or PBAF). For example, FIG. 23 illustrates an embodiments in which ABA and rapamycin induce epigenetic editing though P300, TET3, and a BAF chromatin remodeling complex assembled around BRG1.

This disclosure also describes DNA constructs that encode the components of the CRISPR/CIP system and cells that include those DNA constructs. The constructs generally include one or more exogenous polynucleotides that encode polypeptide components of the CRISPR/CIP platform. The exogenous polynucleotides may be introduced into a cell individually. Alternatively, two or more exogenous polynucleotides may be provided in a single vector.

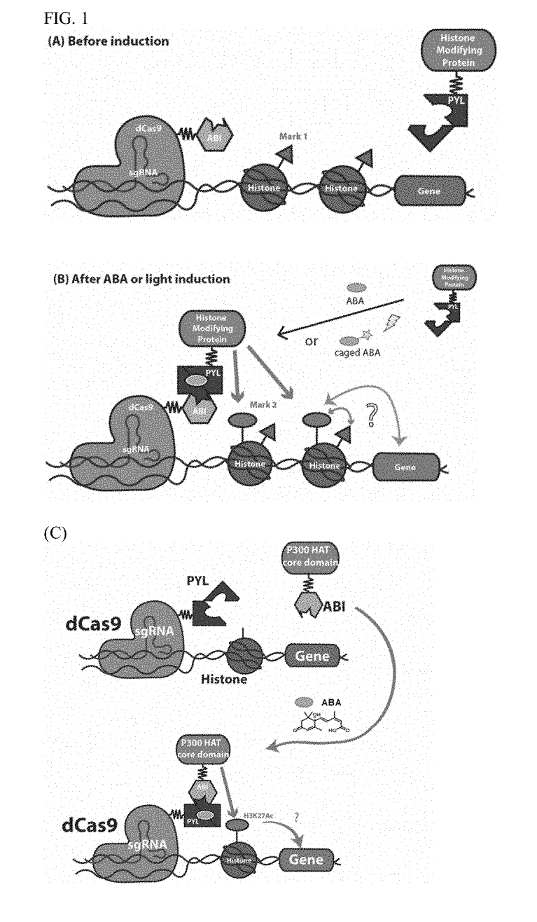

Thus, one can, for example, link PYL to a histone-modifying protein and separately link ABI to dCas9, as shown in FIG. 1A. ABA can be used to temporally control association between the histone-modifying protein and dCas9, which can to be targeted to a defined genome locus when paired with a sequence specific sgRNA (FIG. 1B). To recruit two histone-modifying proteins to the same locus simultaneously or sequentially, one can link dCas9 to both ABI and FKBP, and have PYL and FRB individually fused to unique histone-modifying proteins. Upon adding ABA and/or Rap, these two histone-modifying proteins can be independently recruited to the chosen genome locus with temporal control—i.e., one can control the order and timing of each histone-modifying protein to the chosen genome locus. If more histone-modifying proteins need to be recruited, an additional CIP system—e.g., using gibberellic acid (GA) as an inducer—can be used. These—and other—CIP systems can be activated by providing a chemical inducer and/or by light.

In another example, PYL can be fused to dCas9 and ABI can be fused to a histone-modifying protein (e.g., the catalytic acetyltransferase (HAT) domain of P300, which acylates H3K27), as illustrated in FIG. 1C. By linking PYL to dCas9 and linking ABI to the P300 HAT domain, P300 HAT can be recruited by ABA to specific genome loci targeted by customized sgRNAs. Although chemical inducible systems for similar purposes have been reported, they rely on genome-targeting methods that are less easily customized. Consequently, this new system, employing a readily customizable dCas9-based platform, facilitates customizable epigenome editing. The reversible nature of the ABA system allows the rapid release of the recruited P300 HAT, which enables one to monitor the stability and memory of the artificially established epigenetic environment.

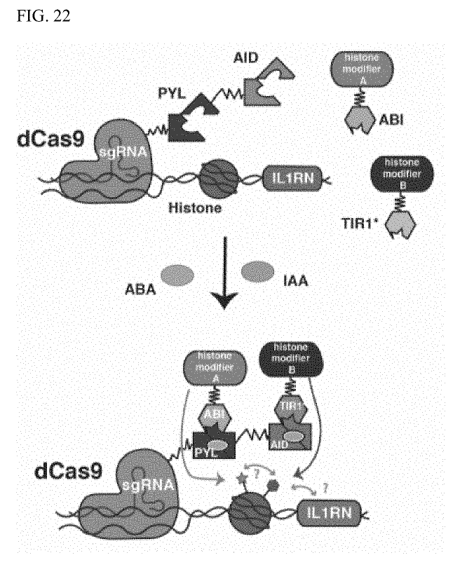

In other examples, illustrated in FIG. 22 and FIG. 23, the system can be designed to involve two inducers. FIG. 22 shows a system in the context of histone-modification epigenetic editing. FIG. 23 shows a system in the context of recruiting DNA demethylation enzyme and a chromatic remodeling complex. As illustrated in FIG. 23, a two-inducer system can be designed so that each inducer can induce a different epigenetic activity. Moreover, the system can be activated using either inducer individually, using both inducers simultaneously, or using the two inducers sequentially.

The technology described herein can be generally applied to recruit any chosen chromatin-modifying protein—e.g. a histone tail PTM modifying enzyme, a chromatin remodeling protein or complex, or a DNA methylation and de-methylation enzyme—to any chosen genome locus. This method enables a researcher to investigate epigenetic modifications in their native environment—i.e., in living cells—in a new and effective way. This technology can potentially be developed for therapeutic purposes, which allows the editing and correction of the erroneous epigenome associated with specific human diseases.

Most of the current methods to study the roles of histone posttranslational modifications (PTMs) rely on the overexpression or knockdown of specific histone-modifying proteins. These approaches usually lead to global changes in the epigenetic landscape that make it difficult to differentiate if the observed effects on gene activities are due to changes in local epigenetic environments or instead are secondary effects caused by remote changes in gene activities. These methods also are not reversible, do not allow precise temporal control that can allow one to study the order of events, and/or face other limitations. For example, chromatin-modifying proteins are fused to DNA binding domains (DBDs) of transcription factors (TFs) that recognize unique DNA sequences that either already exist in the genome (e.g., NF1-DBD targets the endogenous c-fos promoter), or are inserted at specific genome loci (e.g. GAL4-DBD targets the inserted exogenous UAS sequence). However, these approaches are limited either by the number of the existing loci that DBDs can recognize in the genome or require significant efforts to knock-in the DBD-binding sequences to the chosen genome loci. Other recent methods use zinc finger proteins (ZFPs) or transcription activator-like effectors (TALEs) as DBDs that can be engineered to potentially target any endogenous DNA sequence in the genome. Light-dimerizable protein domains can also be incorporated to achieve light-inducible recruitment of different histone-modifying proteins using TALE-DBDs. However, the binding specificity of these ZFPs and TALEs is usually degenerate and the time-consuming design and generation limits their general application. All of the above methods have their limitations for the studies of epigenetic modifications.

Compared to the overexpression or knock-down methods to study epigenetic modifications, the CRISPR/CIP method described herein can achieve the same purposes by recruiting proteins that write or erase specific histone PTMs. The method described herein can achieve the “localized” editing that re-writes histone PTMs only at chosen genome loci and/or allow direct observations of effects on gene activity due to local epigenetic changes. It significantly eliminates the complication and uncertainty in interpreting observed effects that can also be caused indirectly in overexpression/knock-down methods.

Also, compared to methods that allow localized editing of the epigenome—e.g., using TF-DBDs, ZFPs, or TALEs as targeting domains—the CRISPR/dCas9 targeting method is simple to design and implement without the need to engineer customized DBDs or insert DBD-binding sequences into defined genome loci. It offers high flexibility in the choice of genome loci and genes to study.

Also, the Tet-ON inducible system can be used to induce the expression of dCas9 fusion proteins using doxycycline. However, the CIP-based induction is much faster and can provide higher degree temporal control to better dissect the dynamic roles of histone posttranslational modifications (PTMs). Inducible systems that control at the transcription level—e.g., the Tet-ON system—need to go through the transcription and translation processes to produce proteins, which takes extra time. To remove the activity of the induced proteins, these proteins have to be degraded and the duration will depend on their cellular half-lives. In contrast, the CIP-based method described herein has all the protein components already produced in cells that can readily be dimerized to induce effects. Once the inducer is removed (e.g., by a simple wash), the dimerized proteins will dissociate and chromatin-modifying proteins will be released from the genome loci and no longer be effective.

This new method is highly modular, enabling customizability to design the most suitable platforms to answer a specific question. For example, the CRISPR/CIP method can be tailored to recruit specific combinations of histone-modifying proteins to create a specific set of histone PTMs simultaneously or sequentially by using orthogonal CIP inducers. Other epigenetic modifying proteins—e.g., chromatin remodeling complexes or DNA methyltransferases—also can be used to replace histone-modifying proteins for other types of epigenetic editing. The epigenetic landscape at multiple genome loci can be edited at the same time by using multiple sgRNAs designed for different genes. The orthogonal CIP inducers can be caged to become activatable by different wavelengths of light to achieve a high degree of spatiotemporal regulation, especially for studies in multicellular environments—e.g. in tissues or an organism.

Epigenetic dysregulation has been linked to the pathogenesis of various cancers caused by altered expression patterns of regulatory genes such as, for example, tumor suppressors. One of the therapeutic strategies in cancer treatment is to correct erroneous epigenetic environments by targeting histone/chromatin-modifying enzymes by, for example, using HDACs inhibitors. These epigenetic inhibitors generally exhibit a lack of specificity in inhibiting a selective protein target and are unable to achieve gene-specific regulation.

The CRISPR/CIP technology described herein can be a powerful tool for epigenetic research and also can be transformed into a new cancer therapy by correcting erroneous local epigenetic environments of regulatory genes such as, for example, tumor suppressors. Persistent activation or silencing of targeted tumorigenesis regulators can be achieved to reverse the course of tumor formation or progression. Epigenetic editing events can be controlled by, for example, the pro-drug version of CIP inducers (e.g., ABA pro-drugs) that can be activated either by endogenous local tumor-specific signals or exogenously applied light-irradiation to achieve tumor-selective or spatial-specific cancer treatment.

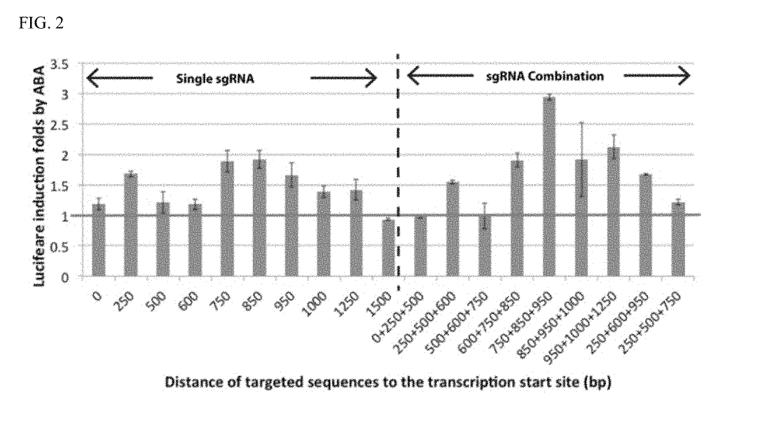

In one exemplary embodiment, ABA-induced ABI-PYL dimerization recruited a transcriptional activator to a DNA sequence targeted by sgRNAs and dCas9, and turn on the expression of a reporter gene. Several DNA plasmids were constructed for this purpose, including: (i) a DNA plasmid (PYL-VP80-ires-dCas9-NLS-ABI) encoding the transcriptional activator VP80 fused to PYL (PYL-VP80) and dCas9 with a nuclear localization signal (NLS) fused to ABI (dCas9-NLS-ABI); (ii) a luciferase reporter gene (fos-Luc) that contains the fos minimal promoter to give minimal background expression; and (iii) several sgRNA expression constructs to target the upstream region (within 1.5 kb) of the luciferase gene. After transfecting these DNA plasmids into CHO cells for 24 hours, we observed that upon adding ABA (100 μM for 16 hours), luciferase expression was induced (FIG. 2). Also, specific combinations of multiple sgRNAs increased the induced expression level. In other applications, the fold induction can be improved by optimizing the targeting region of sgRNAs, the combination of different sgRNAs, and/or the framework of fusion proteins, which may lead to a better positioning of the recruited transcription machinery relative to the transcription start site. These results demonstrate the feasibility of using integrated CRISPR/CIP technology to recruit a desired protein domain to chosen DNA sequences and produce biological outcomes. Since histone posttranslational modifications are generally present in an expanded region instead of a specific location (e.g., a transcription start site), the precise positioning may not be as critical for epigenome editing.

Light can be used to activate caged ABAs and control downstream cellular processes. For example, ABA can be caged with photo-labile groups (e.g. 4,5-Dimethoxy-2-nitrobenzyl, DMNB; or (7-Diethylaminocoumarin-4-yl)methyladenosine-3′,5′-monophosphate, DEACM) and its activity controlled by light. The uncaging process is rapid—e.g., within 1 minute—and caged ABAs are stable in cells for at least 24 hours. These results provide the technical basis for spatiotemporal editing of epigenetic marks. When combined with CRISPR/CIP systems employing other caged inducers—e.g., caged Rap and/or caged GA—one can edit multiple epigenetic marks in a spatiotemporal-specific using light.

Another exemplary embodiment provides for the ABA-inducible modification of H3K27Ac at a specified location. DNA constructs were introduced into HEK 293 cells. The constructs encoded (i) sgRNAs targeting IL1RN gene locus in HEK 293 cells and Oct4 locus in CiA ES cells; (ii) dCas9 fusion proteins that bind sgRNAs, including dCas9-NLSₓ₃-PYL-HA, dCas9-NLSₓ₃-PYLₓ₂-HA, dCas9-NLSₓ₃-PYL-FRB-HA which also serve as negative controls; (iii) histone modifying enzyme domains that can be recruited through ABA to dCas9/sgRNA locations, including FLAG-NLSₓ₂-ABI-P300, FLAG-NLSₓ₂-ABIₓ₂-P300, FLAG-NLSₓ₂-ABI-SUV39H1 and V5-NLSₓ₂-FKBP-SET1; and (iv) constitutively active fusion proteins dCas9-NLSₓ₂-P300-HA and GAL4DBD-NLSₓ₂-SUV39H1 as positive controls.

These constructs allow one to test (i) the targeting of dCas9 fusion protein to IL1RN locus in the presence of corresponding sgRNAs, (ii) the recruitment of P300 fusion protein to the same locus in the presence of ABA, (iii) the increase of H3K27Ac at IL1RN locus with ABA addition, and (iv) the activation of IL1RN activation with ABA.

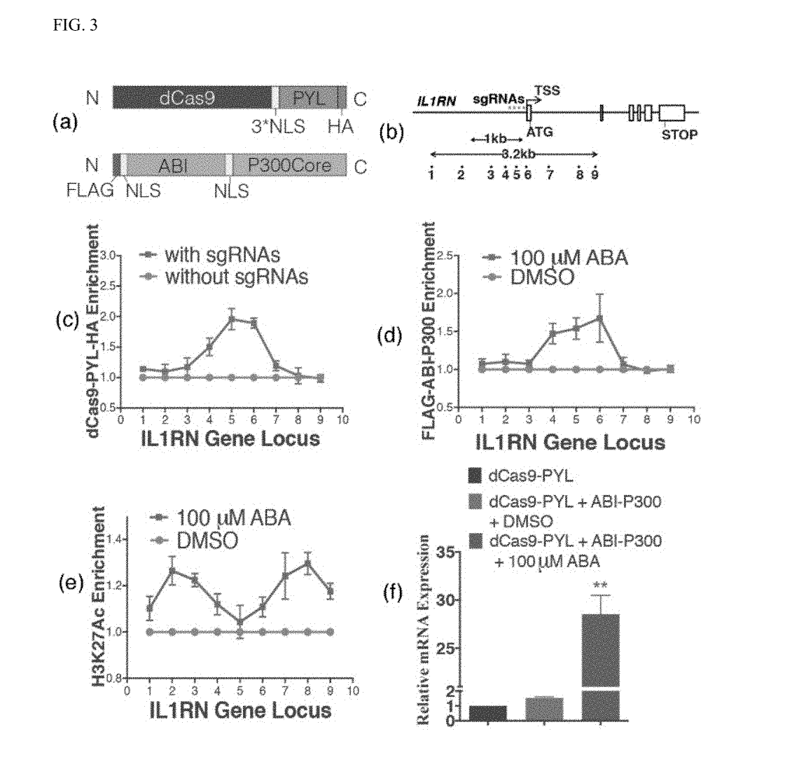

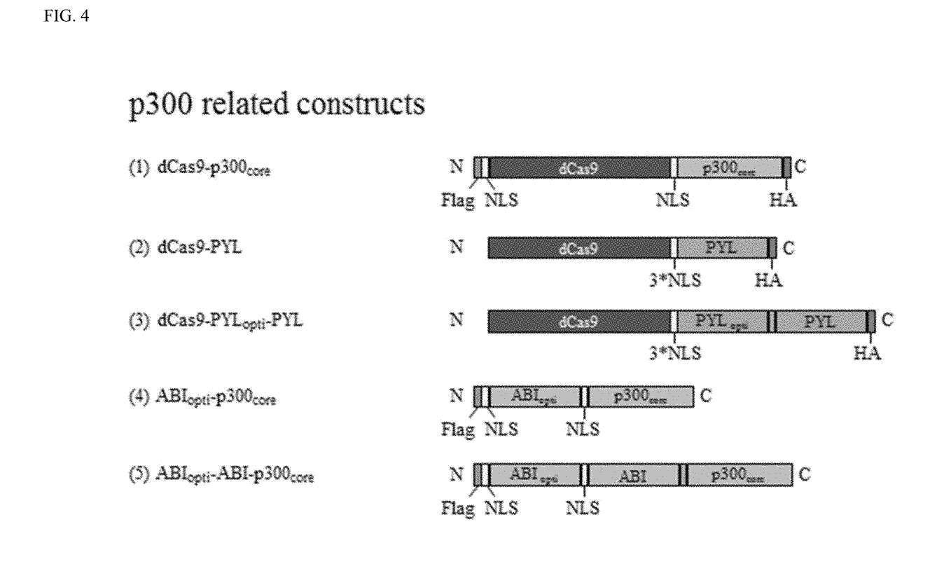

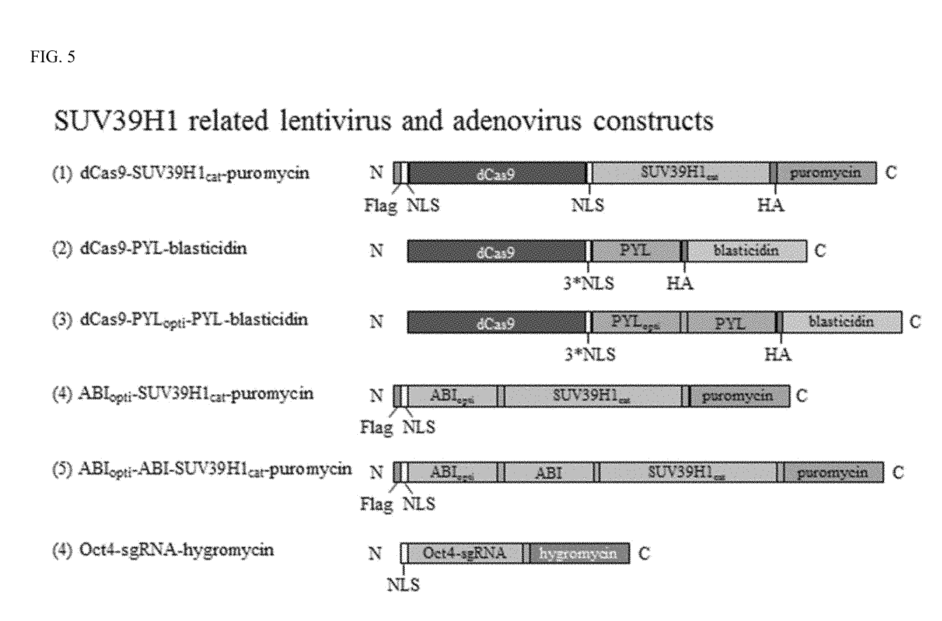

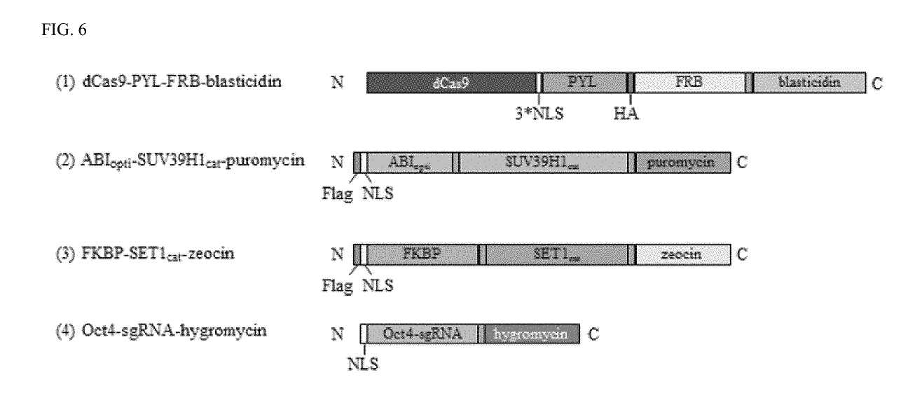

The dCas9-PYL fusion protein can be successfully targeted to IL1RN locus in the presence of sgRNAs, as illustrated in FIG. 1C. To show that dCas9-PYL-HA is targeted to the IL1RN locus, HEK293T cells were transfected with plasmids encoding dCas9-PYL-HA (FIG. 3A) with or without the co-transfection of IL1RN-specific sgRNAs (FIG. 3B) and cultured for 24 hours. Cells were then harvested and subjected to ChIP (using anti-HA antibody) and qPCR analysis in order to quantify the level of dCas9-PYL-HA at the IL1RN locus. FIG. 3C shows that dCas9-PYL-HA is enriched over a narrow area that coincides with the sgRNA-binding locus (primer sets 4 to 6) (FIG. 3C).

To examine the recruitment of FLAG-ABI-P300, HEK293T cells were co-transfected with IL1RN sgRNAs, dCas9-PYL-HA and FLAG-ABI-P300 plasmids for 24 hours. Cells were then either treated or not treated with 100 μM ABA for another 24 hours before being harvested. Portions of the cell lysates were analyzed by using ChIP (with anti-FLAG antibody) and qPCR assays to quantify the level of FLAG-ABI-P300. The addition of ABA leads to an increase in FLAG-ABI-P300 level at the IL1RN locus (FIG. 3D). To determine if the level of H3K27Ac changes in response to the FLAG-ABI-P300 localization, portions of the cell lysates produced in the above experiment were subjected to ChIP (using anti-H3K27Ac antibody) and qPCR assays to quantify the H3K27Ac enrichment. The results show that H3K27Ac levels increase at the IL1RN locus 24 h after ABA addition, and that this increase is not observed in lysates of cells that were not treated with ABA (FIG. 3E).

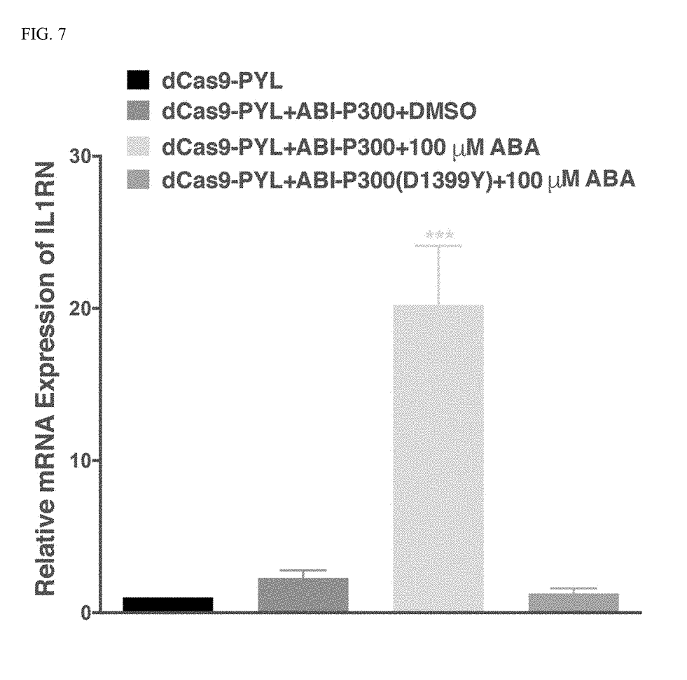

To study how IL1RN expression levels change, total RNAs from the cell lysates generated in the above experiment were purified and then subjected to qPCR analysis to quantify mRNA levels. FIG. 3F shows that IL1RN mRNA level from cells transfected with both ABI-P300 and dCas9-PYL is similar to the level of the one from cells transfected with dCas9-PYL only. This finding suggests that the overexpression of the P300 HAT without localizing to the IL1RN locus did not lead to IL1RN expression. On the other hand, the IL1RN mRNA level increases when transfected cells were treated with ABA (FIG. 3F), indicating that the increase in H3K27Ac activates IL1RN expression. Furthermore, when a P300 mutant (D1399Y) lacking the acetyltransferase activity was used, the IL1RN mRNA level did not increase upon ABA addition (FIG. 7), indicating that IL1RN expression directly depends on H3K27 acetylation. The observations described above demonstrate that the ABA-inducible system enables installation of H3K27Ac at the IL1RN locus and that the consequent increase in acetylation leads to an increased level of IL1RN expression.

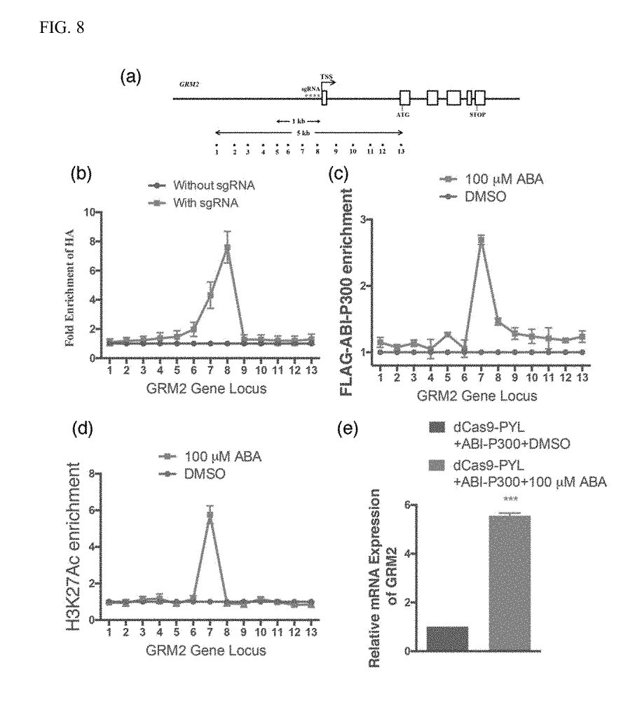

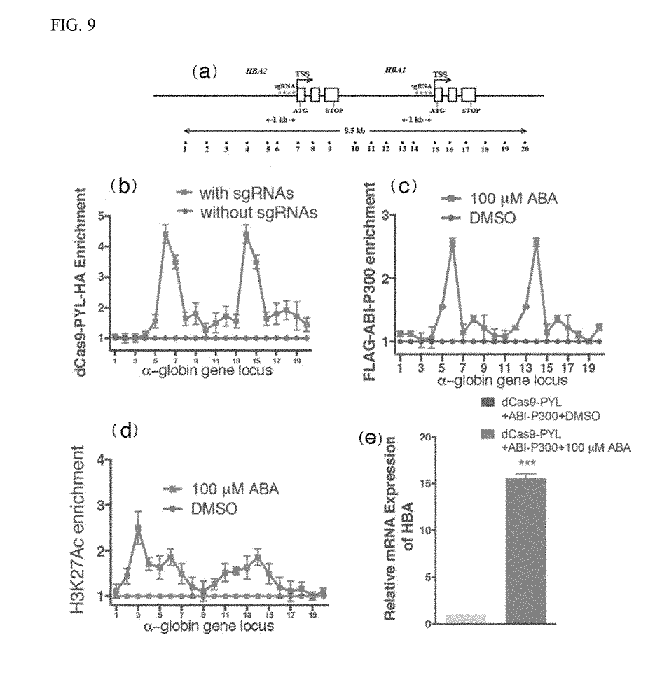

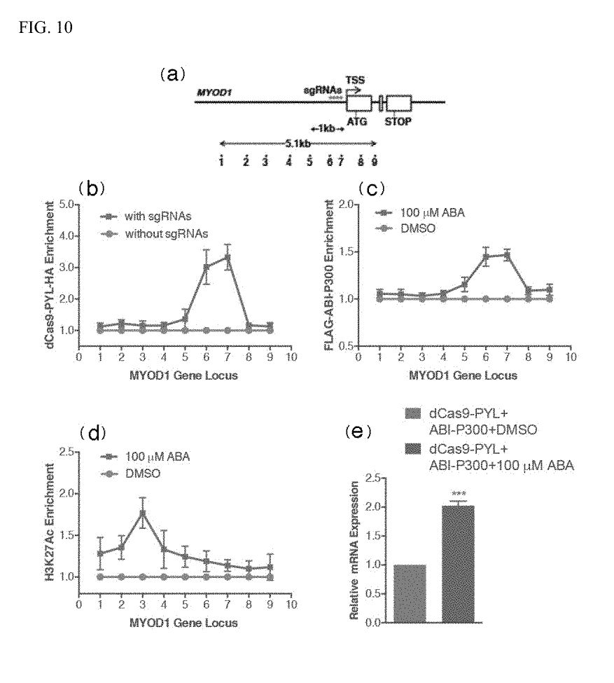

To determine if the new method can be used to modify H3K27Ac at other gene loci, sgRNAs were designed that target the promoter region of other genes with low expression levels in HEK293T cells, including GRM2, HBA and MYOD1 (Table 1). The results of ChIP-qPCR assays employing primers designed to cover these gene loci (FIG. 8A, FIG. 9A, FIG. 10A) show that in the presence of the corresponding sgRNAs, dCas9-PYL-HA is enrichment at each targeted gene locus (FIG. 8B, FIG. 9B, FIG. 10B) and ABA addition causes the enrichment of FLAG-ABI-P300 (FIG. 8C, FIG. 9C, FIG. 10C) and H3K27Ac (FIG. 8D, FIG. 9D, FIG. 10D). The level of mRNAs in each case also increases correspondingly (FIG. 8E, FIG. 9E, FIG. 10E).

It is known that sgRNA/Cas9 can bind to unintended loci, which can lead to off-target effects. To examine if aberrant binding interferes with the interpretation of results, all reported sites in the genome that could potentially be targeted by the IL1RN sgRNAs used in the studies were checked. Changes in the mRNA levels of six genes having TSS close to the off-target sites were monitored (Table 3) after cells were co-transfected with IL1RN sgRNAs, dCas9-PYL-HA and FLAG-ABI-P300 plasmids followed by ABA treatment for 24 hours.

TABLE 3
Off-target binding sites of IL1RN sgRNAs

| | | | Coordinate | | | Base pairs of | | | |
|---|---|---|---|---|---|---|---|---|---|
| Off-Target DNA Position | | | of Closest | | | sgRNA Matches | | | |
| Chr | Start | Stop | TSS | Gene ID | Gene Name | #1 | #2 | #3 | #4 |
| chr1 | 113243341 | 113243689 | 113243748 | NM_001042678 | RHOC | 11 | 11 | 12 | 9 |
| chr1 | 234745953 | 234746504 | 234740014 | NM_001077397 | IRF2BP2 | 10 | 10 | 12 | 8 |
| chr10 | 60144983 | 60145580 | 60144902 | NM_001270782 | TFAM | 11 | 10 | 9 | 9 |
| chr16 | 30996373 | 30996953 | 30996518 | NM_001142777 | HSD3B7 | 12 | 15 | 10 | 11 |
| chr17 | 60500901 | 60501397 | 60501245 | NM_181725 | METTL2A | 11 | 9 | 11 | 11 |
| chr20 | 34042922 | 34043252 | 34043222 | NM_007186 | CEP250 | 11 | 12 | 10 | 11 |

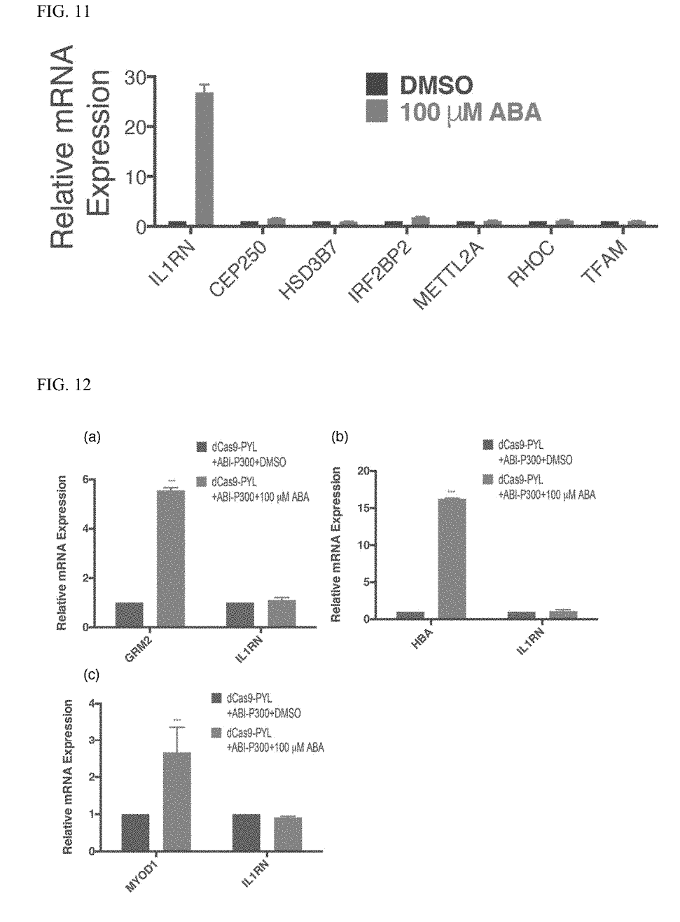

None of the six genes display increases in mRNA levels (FIG. 11) indicates that the off-target acetylation and gene activation are not significant in the studied cases. In addition, when sgRNAs designed for GRM2, HBA or MYOD1 were used in place of those for IL1RN, no increase in IL1RN mRNA was observed (FIG. 12), which demonstrates that the H3K27Ac enrichment at the IL1RN locus and gene expression are specific to IL1RN sgRNAs.

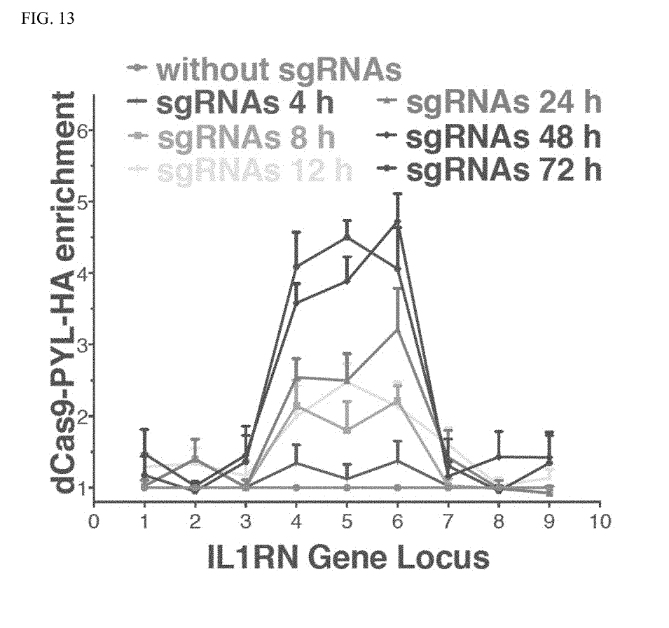

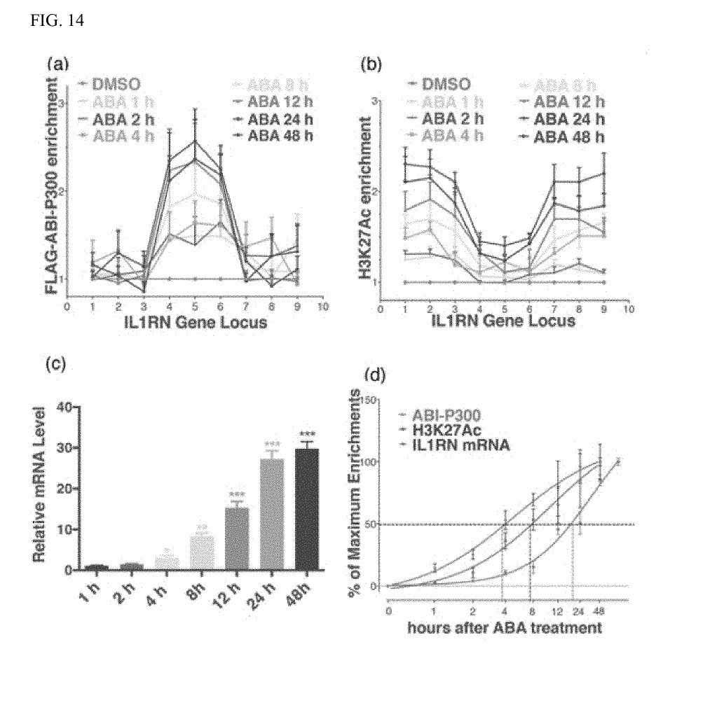

In some embodiments, this inducible system allows one to exercise temporal control of gene expression, which enables precise monitoring of the occurrence and progression of histone modification and gene expression events. To demonstrate this feature, the relative timing of changes occurring in the levels of localized FLAG-ABI-P300, enriched H3K27Ac, and expressed IL1RN mRNA after ABA induction were examined. There is a significant level of sgRNA-dependent enrichment of dCas9-PYL-HA at the IL1RN locus at 24 hours after transfection, which is the time point as which ABA was first added (FIG. 13). Within one hour after ABA addition, FLAG-ABI-P300 is rapidly recruited to the IL1RN locus where it reaches a maximal level after 12 hours (FIG. 14A). Correspondingly, the level of H3K27Ac begins to rise one hour after ABA addition and continues to increase until 48 hours (FIG. 14B). Finally, the mRNA level increases four hours after ABA addition and continues to increase rapidly until about 24 hours after ABA is added (FIG. 14C).

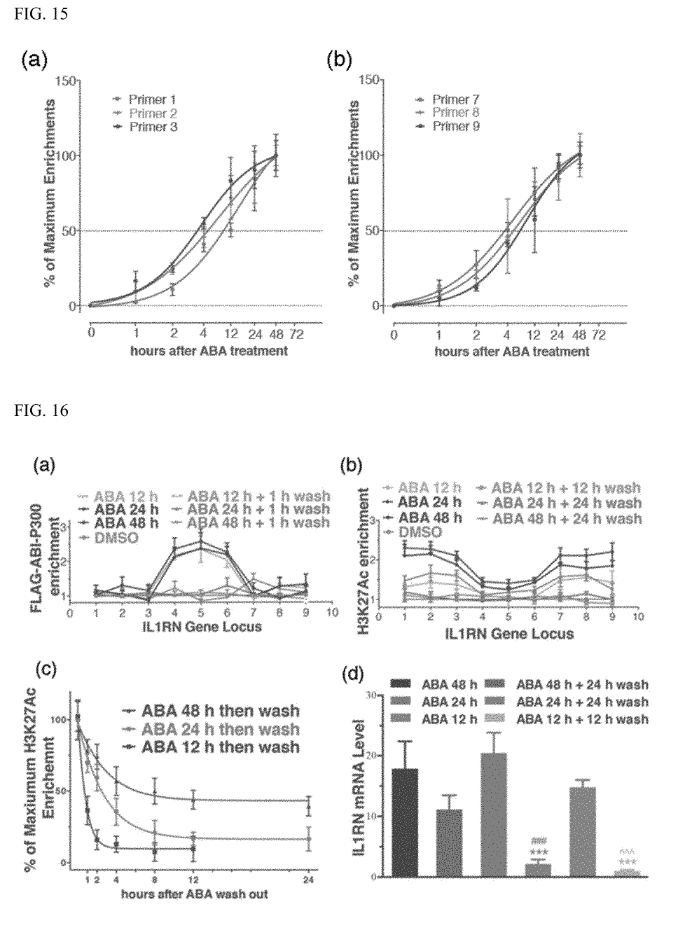

The time-dependent increases of IL1RN mRNA, and enrichments of FLAG-ABI-P300 and H3K27Ac at each of the probed loci were subjected to nonlinear regression analysis in order to elucidate the order of FLAG-ABI-P300 localization, H3K27Ac enrichment, and IL1RN expression. A comparison of the time periods required to reach 50% maximum enrichments shows that FLAG-ABI-P300 localization takes place before H3K27Ac enrichment and that these events are followed by IL1RN mRNA expression (FIG. 14D). The same analysis for the enrichments of H3K27Ac at each probed locus revealed that a longer time is required for the level of H3K27Ac to increase at loci farthest from the FLAG-ABI-P300 site (primer 1 and 9) than the closer sites (primer 3 and 7) (FIG. 15). This finding suggests a possibly spreading mechanism for H3K27Ac installation from the nucleation site at the IL1RN locus.

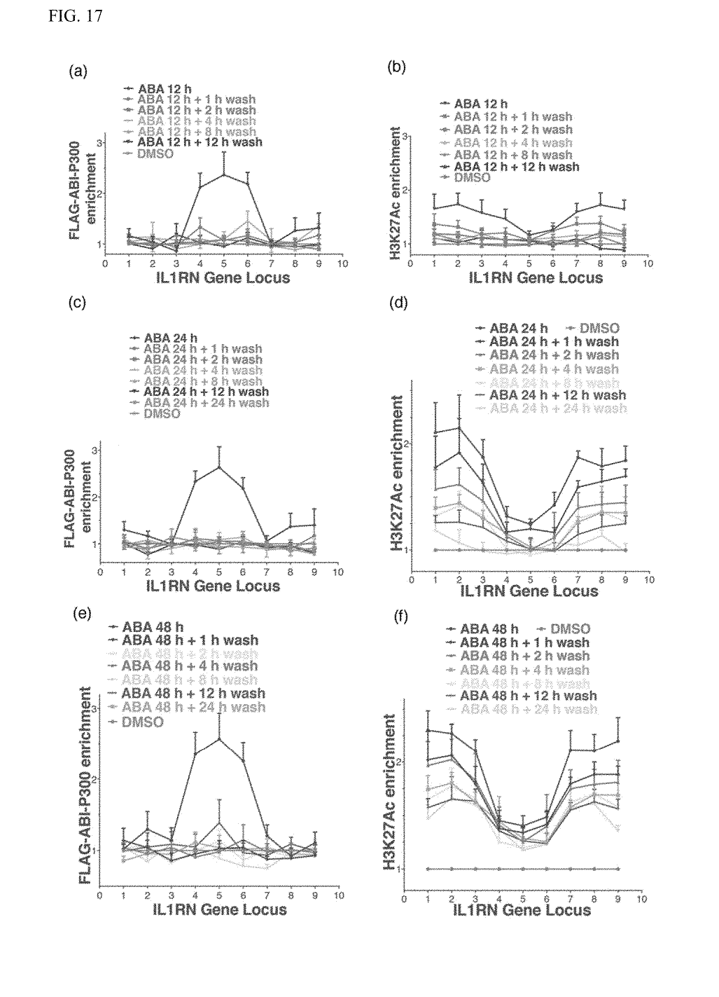

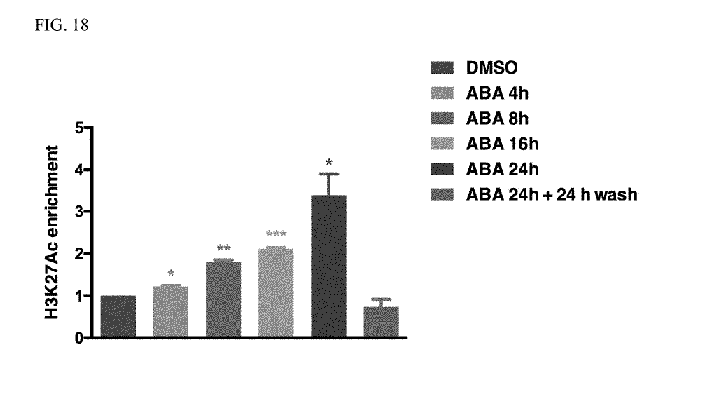

In some embodiments, the system can be designed to be reversible, which can be employed to remove an inducing factor rapidly so that the stability of artificially created histone posttranslational modifications (PTMs) can be monitored in a temporal specific manner. To determine the stability of H3K27Ac installed at the IL1RN locus after FLAG-ABI-P300 removal, HEK293T cells were co-transfected with IL1RN sgRNAs, dCas9-PYL-HA and FLAG-ABI-P300 plasmids for 24 hours and then treated with ABA for another 12 hours, 24 hours, or 48 hours. Cells were then washed with a non-ABA containing media (10 minutes for 3 times, or not washed as negative control) and then subjected to another one-hour to 24-hour culturing period. Cells were then harvested at different time points and assayed using ChIP and qPCR to monitor the levels of FLAG-ABI-P300, H3K27Ac, and IL1RN mRNA. FIG. 16 shows that FLAG-ABI-P300 rapidly decreases to a near background level within one hour following washing under all treatment conditions (FIG. 16A, FIG. 17). However, we observed that a longer ABA incubation time (48 hours) increases the stability of H3K27Ac (FIG. 16B). A comprehensive time course analysis of H3K27Ac levels after ABA removal revealed that the longer cells were treated with ABA, the slower H3K27Ac was removed after washing (FIG. 16C, FIG. 17). Finally, changes of IL1RN mRNA levels after washing under conditions described above are well correlated with the changes in H3K27Ac levels (FIG. 16D), which further confirms the causal relationship between H3K27Ac and IL1RN activation. Further, FIG. 18 shows data demonstrating the reversibility of H3K27Ac at the GRM2 locus. H3K27Ac enrichment was rapidly induced upon ABA addition and reversed after ABA removal.

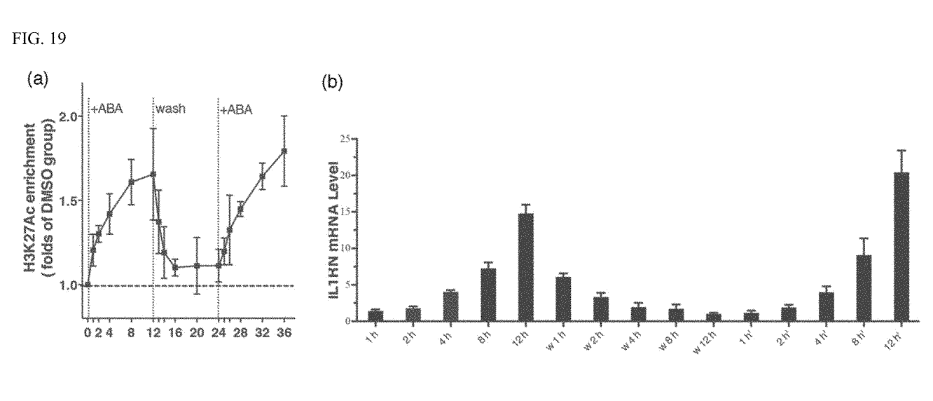

The last phase of this effort was aimed at demonstrating precise temporal control in histone posttranslational modification (PTM) editing by this method. For this purpose, HEK293T cells were co-transfected with IL1RN sgRNAs, dCas9-PYL-HA, and FLAG-ABI-P300 plasmids for 24 hours before adding ABA. Cells were then incubated for 12 hours with ABA, washed, and incubated for another 12-hour period in the absence of ABA. A second dose of ABA was then added to cells, which were incubated for another 12 hours before they were harvested and their H3K27Ac and mRNA levels analyzed. FIG. 19A shows that the H3K27Ac level can be tightly controlled temporally at the IL1RN locus, while FIG. 19B shows that the IL1RN mRNA levels change accordingly.

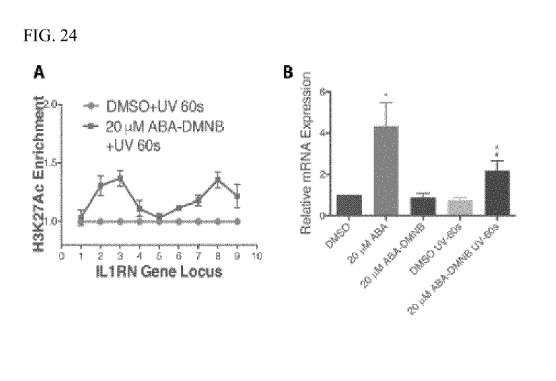

FIG. 24 shows that light H3K27Ac level can be controlled using a light-inducible CIP technology. ABA was chemically modified to include a photo-cleavable group (e.g. 4,5-dimethoxy-2-nitrobenzyl, DMNB or [7-(diethylamino)coumarin-4-yl]methyl, DEACM) that masked ABA dimerization function and could be removed upon irradiation with a specific wavelength of light. Integrating caged ABA into the CIP method, light can be used to rapidly induce downstream effects. Also, when introduced into the inducible epigenetic editing platform, light can be applied to edit H3K27Ac and control transcriptional activation. These studies support the feasibility of proposed light inducible epigenetic editing technology.

In another embodiment, the CRISPR/CIP method can use ABA to recruit the catalytic domain of SUV39H1 and install an H3K9me3 modification at the Oct4 locus in ESCs. In this embodiment, DNA constructs (e.g., lentiviral constructs) can be designed to encode dCas9, the catalytic domain of SUV39H1 that can be dimerized by ABA, and Oct4-specific sgRNAs. One exemplary construct, HA-dCas9-NLS-ABI-blast, contains dCas9, a HA tag (for easy detection), and a blasticidin selection cassette. Also, one can make several Oct4-sgRNA-hydro plasmids that target the promoter region (within 1 kb upstream) of the Oct4 gene and contain a hygromycin selection cassette. Another exemplary construct, FLAG-PYL-SUV39H1_{cat}-puro, encodes the catalytic domain of SUV39H1 (amino acid 82-412) that trimethylates H3K9 and also contains a FLAG tag (for detection) and a puromycin selection cassette. Just the catalytic domain of SUV39H1 was chosen to minimize interactions with other chromatin-associated proteins that may complicate the resulting effects as well as to reduce the size of DNA to be packaged into the viral capsid. Lentivirus can be produced from HEK293T cells using the second generation lentiviral packaging and envelope plasmids (i.e., psPAX2 and pMD2.G) to infect the ESCs. Lentiviral infection, instead of transient transfection, can be used to increase the efficiency of DNA introduction into cells and the rate of genome insertion, which can allow one to follow the changes of histone PTMs and associated gene activities for extended time periods.

The inducible installation of the H3K9me3 PTM at the Oct4 locus in ESCs can be used as a model to test the effectiveness of using the ABA-based CRISPR/CIP inducible system to edit histone PTMs. The Oct4 locus in ESCs is known to have a high level of H3K4me3 and a minimal level of H3K9me3, which is well correlated with the active expression status of the Oct4 gene. ESCs infected with lentiviral constructs described immediately above will be selected, after 48 hours of infection, with corresponding combinations of puromycin, blasticidin, and hygromycin to remove cells without the introduced DNAs. The selected cells in each infection condition will be amplified and then treated with ABA (10 μM to 250 μM) for a different period of time (four hours to 72 hours), or without ABA as a negative control. Cells in each treatment condition can be harvested and processed following the standard chromatin immunoprecipitation (ChIP) protocol. Antibody against H3K9me3 can be used to pull down H3K9me3-associated DNA fragments for analysis by quantitative PCR (qPCR). Anti-HA and Anti-FLAG antibodies also can be used for pull-down to confirm the localization of each fusion protein. Anti-H3 antibody and IgG pull-down can be used as the positive and the negative controls. Previously reported qPCR primers for probing the Oct4 locus can be used in qPCR assays. Using CHIP-qPCR analysis, one can examine whether the dCas9 fusion protein is targeted to the Oct4 locus by the Oct-sgRNAs and if the SUV39H1_{cat} fusion protein is recruited upon adding ABA. One can compare the level of the H3K9me3 modification in different viral infection conditions and different ABA treatment time periods.

Another exemplary embodiment uses ABA-DMNB combined with light, instead of using ABA, to induce the epigenetic modification. The virus-infected and antibiotic-selected cells described immediately above can be treated with ABA-DMNB (100 μM) followed by irradiation with 365-nm light for one minute and then cultured for a different period of time (four hours to 72 hours). Cells can be harvested and analyzed for H3K9me3 level in each treatment condition using the ChIP-qPCR assay. One also can determine if cytotoxicity is associated with the photo-uncaging condition in ESCs based on cell morphology and proliferation rate compared to non-irradiated cells.

Once the H3K9me3 PTM is installed at the Oct4 locus in ESCs, one can determine, for example, whether the H3K4me3 and H3K9me3 marks can co-exist or, conversely whether one of the marks is removed. One also can evaluate, for example, how Oct4 expression changes in response to the changes in these epigenetic marks.

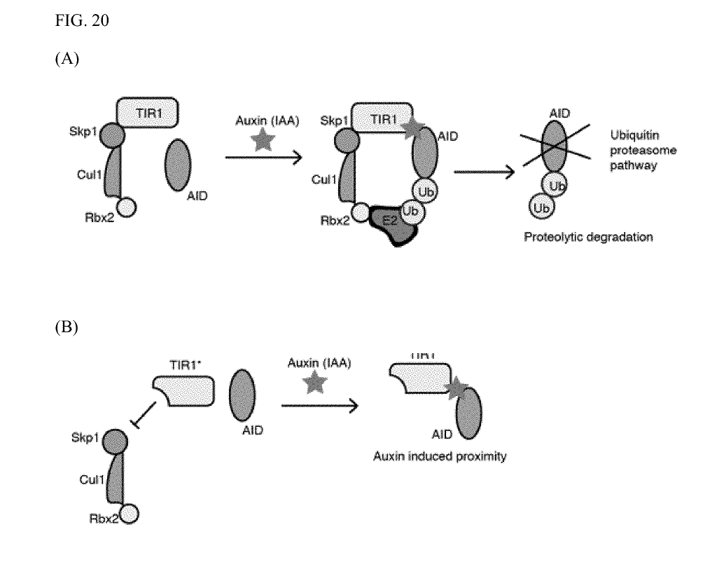

In another embodiment, the CIP system can involve a modification of auxin signaling in plants. Auxin (e.g., indole-3-acetic acid, IAA) is a class of plant hormone that regulates plant development. IAA induces binding between TIR1-coantining proteins and AID-containing proteins, similar to ABA-induced binding between ABI and PYL-fusion proteins. However, in auxin signaling, TIR1 recruits a multi-protein E3 ubiquitin ligase complex (Skp, Cullin, F-box containing complex, SCF complex) that leads to the ubiquitination and degradation of AID proteins. To convert an IAA-induced degradation system into a new IAA-based CIP system, the TIR1 protein was engineered to disrupt the interactions between TIR1 and the SCF complex, while maintaining the IAA-dependent dimerization capability between TIR1 and AID (FIG. 20). TIR1 from Oryza sativa was selected for modification because it possesses thermal stability at the physiological temperature of mammalian cells.

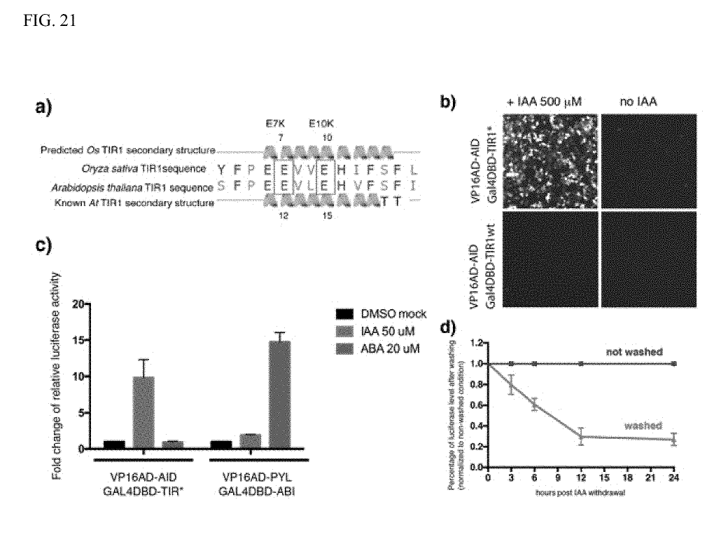

Based on sequence alignment analysis against Arabidopsis thaliana TIR1, a mutant form of Oryza sativa TIR1 (TIR1*) was engineered. TIR1* contained a double mutation, where glutamic acid residues at position 7 and position 10 were replaced with lysine residues (YFPEKVVKHIFSFL, SEQ ID NO:125) that inhibited the ability of TIR* to interact with the SCF complex. To test if TIR1* indeed no longer mediated IAA-induced degradation, but would still bind to AID in the presence of IAA, an IAA-inducible EGFP transcriptional activation assay was constructed. In this assay, EGFP would only be expressed when a functional transcriptional activator was reconstituted by IAA from the two split halves (a DNA binding domain, DBD, and an activation domain, AD, each linked to an IAA-dimerizable protein, TIR* and AID). DNA constructs were made that encoded fusion proteins of GAL4DBD-TIR1*, GAL4DBD-TIR1^{WT} (the mutant (TIR*) and the wild type TIR1 (TIR1^{WT}) fused to DBD from GAL4, respectively) and VP16AD-AID (AID fused to VP16 AD). If the AID fusion protein was degraded or the dimerization of TIR1 and AID was disrupted, no EGFP expression would occur. Either GAL4DBD-TIR1* or GAL4DBD-TIR1^{WT} was co-transfected with VP16AD-AID and an inducible EGFP reporter construct that contains 5×GALDBD binding sites into HEK293T cells with or without the addition of IAA for 24 hours. Only the combination of the TIR1 mutant TIR* and the IAA addition induced the expression of EGFP (FIG. 21B). This indicated that a working IAA-inducible CIP system was successfully created without degrading the AID fusion protein.

To ensure that IAA and ABA could be used to independently control two histone modification editing events, the orthogonality between these two systems was tested. HEK293T cells were co-transfected with either GAL4DBD-TIR1*/VP16AD-AID (responsive to IAA) or GAL4DBD-ABI/VP16AD-PYL (responsive to ABA), and an inducible luciferase reporter construct. This was followed by treating cells with either IAA or ABA (or DMSO as a control) for 24 hours. Only the proper combination of inducer with corresponding binding proteins could induce luciferase expression (FIG. 21C), suggesting that the IAA and ABA systems can co-exist in a single cell and be induced independently of one another. To test the reversibility of IAA-induced dimerization, in which it was critical to achieve precise temporal control, HEK293T cells were co-transfected with GAL4DBD-TIR1*, VP16AD-AID, and an inducible luciferase expression construct, and then treated cells with 50 μM IAA for 24 hours. Cells were then washed with fresh media without IAA to remove the inducer (or no washing as a control). They were then harvested for luciferase assays after washing. The IAA-induced effect was readily reversible (FIG. 21) at a similar rate as the ABA system.

Thus, this disclosure describes a CIP platform for epigenetic editing. The platform exploits CIP systems using various inducers that induce dimerization of dimerizable proteins that, when dimerized, control epigenetic function through dCas9. Some embodiments of the system provide temporal control and/or reversibility. Generally, a CIP system includes a first polypeptide that forms a complex with a second polypeptide in the presence of an inducer. The first polypeptide also generally includes an epigenetic editing domain. The second polypeptide has a dCas9 domain and a domain that forms a complex with the first polypeptide in the presence of the inducer. Finally, the system includes an sgRNA that targets the dCas9 domain to a selected genomic locus. The epigenetic editing domain can include a histone-modifying domain, a DNA methylating domain, a DNA demethylating domain, or a chromatin remodeling domain.

The system may be introduced into a cell for epigenetic editing of the cell's genome. The system is introduced into the cell by transfecting the cell with one or more polynucleotides that encode the components of the system. Thus, the various components of the system may all be encoded by a single polynucleotide. In other cases, a first polynucleotide may encode a subset of system components, a second polynucleotide may encode another subset of system components, and, as needed, a third polynucleotide and additional polynucleotides may encode additional subsets of system components, as needed, until each component of the system is encoded by a polynucleotide.

As described in detail above, a cell may include more than one CIP system. In such embodiments, components of the first CIP system may be encoded by a polynucleotide that encodes one or more components of an additional CIP system. Alternatively, the two CIP systems may be encoded by separate polynucleotides. As illustrated in FIG. 22 and FIG. 23, embodiments involving more than one CIP system may be designed so that epigenetic editing occurs when either CIP system is induced alone, when both CIP systems are induced simultaneously, when both CIP systems are induced sequentially, or so that both CIP systems must be induced for epigenetic editing to occur.

In another aspect, this disclosure describes a method of performing epigenetic editing of a genomic locus. The method generally includes introducing into cells exogenous polynucleotides to produce a modified cell. The exogenous polynucleotides encoding the various components of a CIP system as described herein. Two or more of the exogenous polynucleotides can be included in a single larger polynucleotide or may be introduced into the cell separately. The method further includes contacting the modified cells with the induce in an amount effective to induce the first polypeptide to form a complex with the second polypeptide, then allowing the sgRNA to target the genomic locus, thereby delivering the epigenetic editing domain to the genomic locus. Finally, the method includes allowing the epigenetic editing domain to modify a histone, thereby performing epigenetic editing of the genomic locus.

In the preceding description and in the claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Reagents and DNA Plasmids Construction

ABA was purchased from Gold Biotechnology (St. Louis, Mo.; A-050-5). Polyethylenimine (PEI, Polysciences, Inc., Warrington, Pa.) was used as transfecting agent. All DNA fragments were amplified by Polymerase chain reaction (PCR) from other intermediate constructs with the enzyme of Phusion High-Fidelity DNA Polymerase (New England Biolabs, Inc., Ipswich, Mass.) under S1000 thermal cycler (Bio-Rad Laboratories, Inc., Hercules, Calif.). Plasmid FLAG-dCas9-p300 Core-HA was purchased from Addgene (Watertown, Mass.; plasmid #61357). This plasmid encodes S. pyogenes dCas9 with C-terminal fusion of human p300 HAT core (aa 1048-1664) driven by CMV promoter and it encodes both Flag-tag at the N terminal and HA-tag at the C terminal on insert. FLAG-ABI-P300 construct was generated from FLAG-dCas9-p300 Core-HA by inserting codon-optimized ABI fragment using SacII and AscI and removing dCas9 and N terminal HA-tag. dCas9-PYL-HA construct was generated from eGFP-PYL-HA which encodes HA-tag at the C terminal on insert. (Liang et al., 2011, Sci Signal 4:rs2). To build this plasmid, the eGFP gene was removed from eGFP-PYL-HA construct using PstI and AscI and inserted dCas9-3×NLS fragment amplified from pdCas9-humanized (plasmid #44246, Addgene, Watertown, Mass.), which expresses a catalytically inactive, human codon-optimized Cas9 gene. sgRNAs were expressed using a lentiviral U6 based expression vector derived from pSico. The backbone plasmid pU6-sgGAL4-1 was purchased from Addgene (Watertown, Mass.; plasmid #46915). The sgGAL4-1 gene was removed from this plasmid using BstXI and XhoI and replaced with sgRNA-containing genes that targeting IL1RN, GRM2, HBA and MYOD1 promoters. The primer sequences are listed in the supplementary Table 1 and Table 2.

TABLE 1
sgRNA sequences

| Target | Protospacer | SEQ ID |
|---|---|---|
| Location | Sequence (5′-3′) | NO: |
| IL1RN sgRNA #1 | TGTACTCTCTGAGGTGCTC | 1 |
| IL1RN sgRNA #2 | ACGCAGATAAGAACCAGTT | 2 |
| IL1RN sgRNA #3 | CATCAAGTCAGCCATCAGC | 3 |
| IL1RN sgRNA #4 | GAGTCACCCTCCTGGAAAC | 4 |
| HBA sgRNA #1 | CCGACAGCGCCCGACCCCAA | 5 |
| HBA sgRNA #2 | CCCCGGGCGAGCGGGATGGG | 6 |
| HBA sgRNA #3 | GCCAGCCAATGAGCGCCGCC | 7 |
| HBA sgRNA #4 | CCCGCGTGCACCCCCAGGGG | 8 |
| GRM2 sgRNA #1 | GGATAGGTAAAGGGGCGCGT | 9 |
| GRM2 sgRNA #2 | GAAGGTCACTGCGCCCCGAC | 10 |
| GRM2 sgRNA #3 | GCGCAGAGCGAGAGCGCTCG | 11 |
| GRM2 sgRNA #4 | GTCTGACTATGGGGCGGAGT | 12 |
| MYOD sgRNA #1 | CCTGGGCTCCGGGGCGTTT | 13 |
| MYOD sgRNA #2 | GGCCCCTGCGGCCACCCCG | 14 |
| MYOD sgRNA #3 | CTCCCTCCCTGCCCGGTAG | 15 |
| MYOD sgRNA #4 | AGGTTTGGAAAGGGCGTGC | 16 |

TABLE 2
Primers used for amplification of ABI and dCas9-3 × NLS.

| Target | Forward Primer (5′-3′) | SEQ ID NO: |
|---|---|---|
| ABI-F | CCGACACCGCGGAGTCCCCCTGTATGGGTTCACC | 17 |
| ABI-R | CCGACAGGCGCGCCCAACTTTGCGTTTCTTTTTCGGGCTA | 18 |
| | GCCTTGGATCCGGCAATTGGCTTCAGGTCCACGACGACGA | |
| | C | |
| dCas9-3 × | GAATTCCTCGAGCTCAAGCTTGCGGGACTGCAGATGGACA | 19 |
| NLS-F | AGAAGTATTCTATC | |
| dCas9-3 × | AATTCGTCTTGAGTTGGCGCGCCCCCGGTAGAATTTACCT | 20 |
| NLS-r | TTCT | |

### Cell Culture and Transfection

Human embryonic kidney cell HEK293T (American Type Culture Collection, Manassas, Va.) was cultured in Dulbecco's modified Eagle's medium (DMEM) (Gibco) with 10% FBS (Atlanta Biologicals), 1× GlutaMax (Gibco, Thermo Fisher Scientific, Inc., Waltham, Mass.), and 1× penicillin/streptomycin (Pen/Strep; Gibco, Thermo Fisher Scientific, Inc., Waltham, Mass.). Cells (100,000/ml) were plated in 24-well or 10-cm cell culture plate the day before transfection. For each well of 24-well plate transfection, 500 ng of dCas9-PYL-HA, 420 ng of FLAG-ABI-P300, and a total amount of 200-ng mix of gRNAs, with a 50-ng per each gRNA, were mixed with 50×(v/w) Opti-MEM (Gibco, Thermo Fisher Scientific, Inc., Waltham, Mass.), then incubate with the same volume of 3 μg PEI-Opti-MEM solution for 20 minutes at room temperature. Then, 100 μL of PEI-combined plasmids were added to every single well of 24-well plate and cultured for 24 hours before experiments were performed. For a 10 cm plate, 1 mL of PEI-combined plasmids were needed. After six hours transfection, cells were washed and incubated for another 1-72 hours before experiments were performed.

### Western Blot Analyses

Cells were washed twice with ice-cold PBS, and lysed with ice-cold 1×RIPA buffer (Cell Signaling Technology Co., Danvers, Mass.; #9806) containing protease inhibitors (AMRESCO, M222) for 30 minutes on ice. The lysates were centrifuged at 16,000×g for 20 min in a 4° C. precooled centrifuge. The protein concentration was determined based on the Bradford method (Bio-Rad Laboratories, Inc., Hercules, Calif.). Equivalent amounts of protein (20 ug) were separated by 4-15% precast polyacrylamide gel (Bio-Rad Laboratories, Inc., Hercules, Calif.) and transferred to a polyvinylidene difluoride membrane (PVDF, Bio-Rad Laboratories, Inc., Hercules, Calif.). The membranes were blocked by TBST buffer (150 mM NaCl, 50 mM Tris, (pH7.6), 0.1% (v/v) Tween 20) supplemented with 5% nonfat dry milk at room temperature for two hours. Then the membranes were incubated overnight with anti-Flag antibody (F7425, 1:2000, Sigma-Aldrich, St. Louis, Mo.) or anti-HA antibody (#26183, 1:5000, Thermo Fisher Scientific, Inc., Waltham, Mass.) or anti-GAPDH antibody (#5174, 1:2000, Cell Signaling Technology, Co., Danvers, Mass.) at 4° C. After three times washing with TBST buffer, the membranes were incubated with horseradish peroxidase (HRP)-conjugated secondary antibodies (anti-rabbit IgG, #7074, 1:2000, Cell Signaling Technology, Co., Danvers, Mass.) for one hour at room temperature. The protein bands were visualized with ECL detection reagents (Bio-Rad Laboratories, Inc., Hercules, Calif.) following exposure to Molecular Imager Gel Doc XR system (Bio-Rad Laboratories, Inc., Hercules, Calif.).

### Chromatin Immuno-Precipitation (ChIP) Assay

ChIP protocol was optimized from the method in Molecular Cloning, Fourth edition (Cold Spring Harbor Laboratory Press, 2012). In brief, cells cultured in a 10-cm plate were fixed with 1 mL of cross-linking buffer (0.1 M NaCl, 1 mM EDTA, 0.5 mM EGTA, 50 mM HEPES and 11% formaldehyde), and shaken in room temperature for 15 minutes. Then 1.1 mL of 1.25 M glycine was added to the solution to stop crosslinking. After washing three times in ice cold PBS, cells were scraped and suspended in 10 mL of lysis buffer (50 mM HEPES, 140 mM NaCl, 1 mM EDTA, 10% Glycerol, 0.5% NP40, 0.25% Triton X-100), and rocked at 4° C. for 10 minutes. Cells were centrifuged at 1,000×g for 10 minutes and were resuspended in protein extraction buffer (200 mM NaCl, 1 mM EDTA, 0.5 mM EGTA, 10 mM Tris). Nuclei were pelleted by centrifuging at 1,000×g for 15 minutes at 4° C., resuspended in 2 mL of chromatin extraction buffer (1 mM EDTA, 0.5 mM EGTA, 10 mM Tris), and divided into six sonication tubes. Then, the samples were sonicated for 24 cycles at a 30 seconds on/30 seconds off protocol. Then samples were moved to a new centrifuge tube and insoluble debris were removed by centrifuge at 2,000×g for 20 minutes at 4° C. 10% volume of samples were stored at −80° C. as 10% input control. Other samples were incubated with anti-Flag M2 affinity gel (A2220, Sigma-Aldrich, St. Louis, Mo.) or anti-HA-agarose antibody (A2095, Sigma-Aldrich, St. Louis, Mo.) or H3K27Ac antibody (07-360, MilliporeSigma, Burlington, Mass.) labeled magnetic beads (26162, Thermo Fisher Scientific, Inc., Waltham, Mass.) for 24 hours at 4° C. Then, sample-bound beads were washed eight times with RIPA buffer (50 mM HEPES, 1 mM EDTA, 1% NP40, 0.7% DOC, 0.5 M LiCl, 1× protease inhibitor complete cocktail), and finally re-suspended in TE (pH 8.0) buffer to wash. Then TE buffer was removed completely, 50 μL of elution buffer (10 mM Tris, 1 mM EDTA, 1% SDS) was added and the samples were incubated for 10 minutes at 65° C. and rocked every two minutes. The liquid was collected, 120 μL of elution buffer was added, and the samples were incubated at 65° C. overnight. Samples of 10% input were incubated with 120 of elution buffer at 65° C. overnight, too. To remove histones, washing buffer (140 μL of TE buffer, 3 μL of 20 mg/ml glycogen and 7 μL of 10 mg/ml Proteinase K) was added and incubated two hours at 37° C. Then ChIP samples were purified with phenol:chloroform:isoamyl alcohol (25:24:1) twice, then purified aqueous in chloroform. The aqueous phase was collected, 700 μL of 100% ethanol was added, incubated 30 minutes at −80° C., and centrifuged at 14,000 rpm for 15 minutes at 4° C. The pellets were washed with 70% ethanol and RNAs were removed with RNase A. At last, DNAs were purified by QIAQUICK purification kit (Qiagen, Hilden, Germany). The primer sequences are listed in the supplementary Table 3.

TABLE 3
ChIP-qPCR primers

| Target | | | SEQ ID NO: |
|---|---|---|---|
| IL1RN-1 | Forward Primer | CCACATGCCAAGCACTTTAC | 21 |
| | (5′-3′) | | |
| | Reverse Primer | GATCCTGGGCCACATCTATAA | 22 |
| | (5′-3′) | | |
| IL1RN-2 | Forward | CCCTGTCAGGAGGGACAGAT | 23 |
| | Reverse | GGCTCACCGGAAGCATGAAT | 24 |
| IL1RN-3 | Forward | TGTTCCCTCCACCTGGAATA | 25 |
| | Reverse | GGGAAAATCCAAAGCAGGAT | 26 |
| IL1RN-4 | Forward | GTTAGAGCGTTGGGGACCTT | 27 |
| | Reverse | CACATGCAGAGAACTGAGCTG | 28 |
| IL1RN-5 | Forward | TTCTCTGCATGTGACCTCCC | 29 |
| | Reverse | ACACACTCACAGAGGGTTGG | 30 |
| IL1RN-6 | Forward | GCTGGGCTCCTCCTTGTACT | 31 |
| | Reverse | GCTGCTGCCCATAAAGTAGC | 32 |
| IL1RN-7 | Forward | CAGGTGAACAGAGAGGTGTAAC | 33 |
| | Reverse | GGCTATTTCACCAATTTCCCTATTC | 34 |
| IL1RN-8 | Forward | CATGGTGAAACCCTGTCTCTATT | 35 |
| | Reverse | CAATTCTCCTGCCTTAGCTACC | 36 |
| IL1RN-9 | Forward | TGTAGTAAGCACAGTGGAAGTG | 37 |
| | Reverse | GAGAGGGTTCAGTGGGATAAAC | 38 |
| HBA-1 | Forward | GAGACCAGCCTGACCAATATG | 39 |
| | Reverse | CCGGGTTCAAGCGATTCT | 40 |
| HBA-2 | Forward | CTGAGGGAACACAGCTACATC | 41 |
| | Reverse | GGAGACACTTCACTGAGAATAGG | 42 |
| HBA-3 | Forward | CCACTACGCCTGGCTAATTT | 43 |
| | Reverse | AGATGCGAGGTCAGGAGAT | 44 |
| HBA-4 | Forward | CCACCACACCCAGCTAATTT | 45 |
| | Reverse | GCCTGTAATCCCAGCACTTT | 46 |
| HBA-5 | Forward | CGGGTAGAGGAGTCTGAATCT | 47 |
| | Reverse | CTAGTGTGCTTGAGTGACAGG | 48 |
| HBA-6 | Forward | ATCCCGCTGGAGTCGAT | 49 |
| | Reverse | GCGCCACCCTTTCCTTT | 50 |
| HBA-7 | Forward | ACAGACTCAGAGAGAACCCA | 51 |
| | Reverse | GCCTCCGCACCATACTC | 52 |
| HBA-8 | Forward | TGGACGACATGCCCAAC | 53 |
| | Reverse | GCCGCTCACCTTGAAGT | 54 |
| HBA-9 | Forward | CCGTGCTGACCTCCAAATAC | 55 |
| | Reverse | GCCCACTCAGACTTTATT | 56 |
| HBA-10 | Forward | CATGCCTGTAAACCCACCTAC | 57 |
| | Reverse | GGAGTGCAATGGTGTGATCTT | 58 |
| HBA-11 | Forward | CAGGGAGCAGGCTGAAG | 59 |
| | Reverse | GGGATGGTACTGAGGAGAAA | 60 |
| HBA-12 | Forward | CCAGGAAGCCCTCAGACTA | 61 |
| | Reverse | CCATCACACAAGTACACACAGA | 62 |
| HBA-13 | Forward | CGGGTAGAGGAGTCTGAATCT | 63 |
| | Reverse | CTAGTGTGCTTGAGTGACAGG | 64 |
| HBA-14 | Forward | ATCCCGCTGGAGTCGAT | 65 |
| | Reverse | GCGCCACCCTTTCCTTT | 66 |
| HBA-15 | Forward | ACAGACTCAGAGAGAACCCA | 67 |
| | Reverse | GCCTCCGCACCATACTC | 68 |
| HBA-16 | Forward | TGGACGACATGCCCAAC | 69 |
| | Reverse | GCCGCTCACCTTGAAGT | 70 |
| HBA-17 | Forward | CCGTGCTGACCTCCAAATAC | 71 |
| | Reverse | GCCCACTCAGACTTTATT | 72 |
| HBA-18 | Forward | ATATGGGTGGTGGGTAGGT | 73 |
| | Reverse | TTTCATCTAGACTGTGCCTGTATC | 74 |
| HBA-19 | Forward | TCTGCCTGCGTTTGTGAT | 75 |
| | Reverse | CAGACCTCCATTCTTCCTGATG | 76 |
| HBA-20 | Forward | GCCTGTAATCCCAGCACTTT | 77 |
| | Reverse | CACCACACCCGGCTAATTT | 78 |
| GRM2-1 | Forward | TACAGATAGGCTGGGAGTGG | 79 |
| | Reverse | CAGGACGTGGAGGATCTCTAA | 80 |
| GRM2-2 | Forward | GTGTGTGTGTGTGTGTGTATTG | 81 |
| | Reverse | CCCATAGAGATGCTGTTAGCC | 82 |
| GRM2-3 | Forward | GGGTGTGCCCATGTGTATTA | 83 |
| | Reverse | CGGATGAGGACATACACTTAGC | 84 |
| GRM2-4 | Forward | AGCCAGAGAGGGTGAGAG | 85 |
| | Reverse | CCCATCACTAACAGGAGACAAG | 86 |
| GRM2-5 | Forward | AGTGTCGGGAATGTGTGTAG | 87 |
| | Reverse | GGGTAGTGGATAAGCTGTAGTG | 88 |
| GRM2-6 | Forward | CCCATTCTCCTCTGACCTCT | 89 |
| | Reverse | CATGTGTCCTCTGCCACTTT | 90 |
| GRM2-7 | Forward | TAAGGCCAGCTCCTCCA | 91 |
| | Reverse | ACTACCACCTAGAGAGCATAGG | 92 |
| GRM2-8 | Forward | GAGTGGGCCACGAAGCA | 93 |
| | Reverse | TCCGCCTCCTCTGGACA | 94 |
| GRM2-9 | Forward | GGGAAGCGGGAAGACAG | 95 |
| | Reverse | CAGACAGAAAGAAGGACCAGAG | 96 |
| GRM2-10 | Forward | GGGTCCTTGAGTTAGTTGCT | 97 |
| | Reverse | CAATCACACAGGCTTGGAAAG | 98 |
| GRM2-11 | Forward | TCCACCTTGCTGTCTCATTC | 99 |
| | Reverse | AGGGTTCTAACCTTTGGTTCTT | 100 |
| GRM2-12 | Forward | CCACTGCTGAGTTTGGTAGG | 101 |
| | Reverse | GGGACAGGATGGTGTCATTAAG | 102 |
| GRM2-13 | Forward | GCACACATCCTCGACAGTT | 103 |
| | Reverse | ATCACCATGGGTCGCATAAG | 104 |
| MYOD-1 | Forward | CCTACTATGCACCTGGCTTTC | 105 |
| | Reverse | GGCATCTCGATGTGTCTTCTT | 106 |
| MYOD-2 | Forward | TGCCTCCAGGTGTAGTAAGA | 107 |
| | Reverse | CCAGCAGCTTCTTGCATTTAG | 108 |
| MYOD-3 | Forward | TCCTAATTGGTCTCCCTCCTT | 109 |
| | Reverse | GCTTGAGCGGAAGAGAAGAATA | 110 |
| MYOD-4 | Forward | AGCAGCAGAGAAGTGAAGAAG | 111 |
| | Reverse | TCTTTGTGCCTTTCCCACTAA | 112 |
| MYOD-5 | Forward | TCGGAGACTTCAGGTGAGAT | 113 |
| | Reverse | GGTGCTGAGTCAAGGAAAGT | 114 |
| MYOD-6 | Forward | GGGACAGAGGAGTATTGAAAGTC | 115 |
| | Reverse | CCCTTTCCAAACCTCTCCAA | 116 |
| MYOD-7 | Forward | TCCTATTGGCCTCGGACT | 117 |
| | Reverse | GCGCCCTGGGCTATTTA | 118 |
| MYOD-8 | Forward | CCGCCTGAGCAAAGTAAATG | 119 |
| | Reverse | CGATATAGCGGATGGCGTT | 120 |
| MYOD-9 | Forward | ATCTCCTACCCACCCGTAAT | 121 |
| | Reverse | AGGCCCGAACACCAATTC | 122 |

### Quantitative RT-PCR Analyses

Total RNA was extracted using the EZNA Total RNA Kit I (Omega Bio-tek, Inc., Norcross, Ga.). qPCR reactions were performed on cDNA prepared from 1,000 ng of total cellular RNA using the High-Capacity cDNA Reverse Transcription Kit (Applied Biosystems Co., Thermo Fisher Scientific, Inc., Foster City, Calif.). TaqMan qPCR probes (Thermo Fisher Scientific, Inc., Waltham, Mass. IL1RN, Hs00893626; GRM2, Hs00968358; HBA, Hs00361191; MYOD1 Hs02330075; GAPDH, Hs99999905) and Fast Advanced Master Mix (Life Technologies Co., Co., Thermo Fisher Scientific, Inc., Carlsbad, Calif.) were used in multiplexed reactions and 384-well format in a 7900HT Fast Real-Time PCR System (Applied Biosystems Co., Thermo Fisher Scientific, Inc., Foster City, Calif.). All measurements were performed at least in triplicate. Data were analyzed using the SDS software, version 2.4 (Applied Biosystems Co., Thermo Fisher Scientific, Inc., Foster City, Calif.), by the ΔΔCt method: the Ct values of target gene were normalized to Ct values of GAPDH, the folds of target gene expression changes were normalized to experimental controls. Analyzed data are reported as the mean±s.e.m. for biological replicates. In ChIP assays, the Ct values of antibodies pulled down segments were normalized to the Ct values of 10% input.

### Statistical Analysis

Data are expressed as mean values±s.e.m. One-way ANOVA or student's t-test statistical analyses, non-liner regression analyses were performed using GraphPad Prism 6.0 software when applicable. Differences were considered significant at P<0.05.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A cell comprising a polynucleotide comprising:
a first exogenous coding region that encodes a first polypeptide that comprises:
a domain that, in the presence of an inducer, forms a complex with a second polypeptide; and
an epigenetic editing domain; and
a second exogenous coding region that encodes the second polypeptide, the second polypeptide comprising:
a domain that, in the presence of the inducer, forms a complex with the first polypeptide;
a dCas9 domain; and
a third exogenous coding region that encodes an sgRNA sequence that targets the dCas9 domain to a genomic locus.

2. The cell of claim 1 further comprising the inducer.

3. The cell of claim 1, wherein the inducer comprises abscisic acid (ABA), gibberellic acid (GA), rapamycin, fusicossin, or auxin.

4. The cell of claim 1, further comprising a second polynucleotide that encodes:
a fourth exogenous coding region that encodes a third polypeptide that comprises:
a domain that, in the presence of an inducer, forms a complex with a fourth polypeptide; and
an epigenetic editing domain; and
a fifth exogenous coding region that encodes the fourth polypeptide, the fourth polypeptide comprising:
a domain that, in the presence of the inducer, forms a complex with the third polypeptide;
a dCas9 domain; and
a sixth exogenous coding region that encodes an sgRNA sequence that targets the dCas9 domain to a genomic locus.

5. The cell of claim 4, wherein the sgRNA encoded by the sixth exogenous coding region is different than the sgRNA encoded by the third exogenous coding.

6. The cell of claim 1, wherein the epigenetic editing domain comprises a histone-modifying domain, a DNA methylating domain, a DNA demethylating domain, or a chromatin remodeling domain.

7. A method of performing epigenetic editing of a genomic locus, the method comprising:
introducing into cells exogenous polynucleotides to produce a modified cell, the exogenous polynucleotides comprising:
a first exogenous coding region that encodes a first polypeptide that comprises:
a domain that, in the presence of an inducer, forms a complex with a second polypeptide; and
an epigenetic editing domain; and
a second exogenous coding region that encodes the second polypeptide, the second polypeptide comprising:
a domain that, in the presence of the inducer, forms a complex with the first polypeptide;
a dCas9 domain; and
a third exogenous coding region that encodes an sgRNA sequence that targets the dCas9 domain to the genomic locus;
providing the inducer in an amount effective to induce the first polypeptide to form a complex with the second polypeptide; and
allowing the sgRNA to target the genomic locus, thereby delivering the an epigenetic editing domain to the genomic locus; and
allowing the epigenetic editing domain to modify the genomic locus.
